(19) United States
(12) Patent Application Publication
(10) Pub. No.: US 2005/0043335 A1
(43) Pub. Date: Feb. 24, 2005

(54) Heterocyclic derivatives of glycinamide and their medical use

(76) Inventors: Richard Leonard Elliott, Essex (GB); Deidre Mary Bernadette Hickey, Hertfordshire (GB); Robert John Ife, Hertfordshire (GB); Colin Andrew Leach, King of Prussia, PA (US); John Liddle, Hertfordshire (GB); Ivan Leo Pinto, Hertfordshire (GB); Stephen Allan Smith, Hertfordshire (GB); Steven James Stanway, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/494,509

(22) PCT Filed: Nov. 8, 2002
(86) PCT No.: PCT/EP02/12505

(51) Int. Cl. C07D 471/02; A61K 31/517; A61K 31/4709; A61K 31/519

(52) U.S. Cl. 514/266.3; 514/300; 514/312; 544/286; 546/122; 546/153; 514/264.1; 544/280

(57) ABSTRACT

Compounds of the formula (I) are inhibitors of the enzyme Lp-PLA [symbol] 2? and are of use in therapy, in particular for treating atherosclerosis.

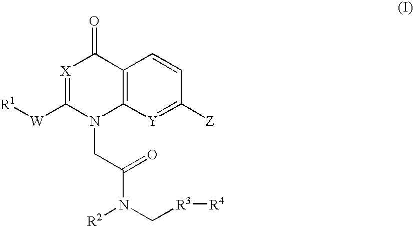

The present invention relates to certain novel compounds, processes for their preparation, intermediates useful in their preparation, pharmaceutical compositions containing them and their use in therapy, in particular in the treatment of atherosclerosis.

WO 95/00649 (SmithKline Beecham plc) describes the phospholipase A₂ enzyme Lipoprotein Associated Phospholipase A₂ (Lp-PLA₂), the sequence, isolation and purification thereof, isolated nucleic acids encoding the enzyme, and recombinant host cells transformed with DNA encoding the enzyme. Suggested therapeutic uses for inhibitors of the enzyme included atherosclerosis, diabetes, rheumatoid arthritis, stroke, myocardial infarction, reperfusion injury and acute and chronic inflammation. A subsequent publication from the same group further describes this enzyme (Tew D et al, Arterioscler Thromb Vas Biol 1996:16;591-9) wherein it is referred to as LDL-PLA₂. A later patent application (WO 95/09921, Icos Corporation) and a related publication in Nature (Tjoelker et al, vol 374, 6 Apr. 1995, 549) describe the enzyme PAF-AH which has essentially the same sequence as Lp-PLA₂ and suggest that it may have potential as a therapeutic protein for regulating pathological inflammatory events.

It has been shown that Lp-PLA₂ is responsible for the conversion of phosphatidylcholine to lysophosphatidylcholine, during the conversion of low density lipoprotein (DL) to its oxidised form. The enzyme is known to hydrolyse the sn-2 ester of the oxidised phosphatidylcholine to give lysophosphatidylcholine and an oxidatively modified fatty acid. Both products of Lp-PLA₂ action are biologically active with lysophosphatidylcholine, in particular having several pro-atherogenic activities ascribed to it including monocyte chemotaxis and induction of endothelial dysfunction, both of which facilitate monocyte-derived macrophage accumulation within the artery wall. Inhibition of the Lp-PLA₂ enzyme would therefore be expected to stop the build up of these macrophage enriched lesions (by inhibition of the formation of lysophosphatidylcholine and oxidised free fatty acids) and so be useful in the treatment of atherosclerosis.

The increased lysophosphatidylcholine content of oxidatively modified LDL is also thought to be responsible for the endothelial dysfunction observed in patients with atherosclerosis. Inhibitors of Lp-PLA₂ could therefore prove beneficial in the treatment of this phenomenon. An Lp-PLA₂ inhibitor could also find utility in other disease states that exhibit endothelial dysfunction including diabetes, hypertension, angina pectoris and after ischaemia and reperfusion.

In addition, Lp-PLA₂ inhibitors may also have a general application in any disorder that involves activated monocytes, macrophages or lymphocytes, as all of these cell types express Lp-PLA₂. Examples of such disorders include psoriasis.

Furthermore, Lp-PLA₂ inhibitors may also have a general application in any disorder that involves lipid oxidation in conjunction with Lp-PLA₂ activity to produce the two injurious products, lysophosphatidylcholine and oxidatively modified fatty acids. Such conditions include the aforementioned conditions atherosclerosis, diabetes, rheumatoid arthritis, stroke, myocardial infarction, ischaemia, reperfusion injury and acute and chronic inflammation.

Patent applications WO 96/12963, WO 96/13484, WO 96/19451, WO 97/02242, WO 97/217675, WO 97/217676, WO 96/41098, and WO 97/41099 (SmithKline Beecham plc) disclose inter alia various series of 4-thionyl/sulfinyl/sulfonyl azetidinone compounds which are inhibitors of the enzyme Lp-PLA₂. These are irreversible, acylating inhibitors (Tew et al, Biochemistry, 37, 10087, 1998).

A further class of compounds has now been identified which are non-acylating inhibitors of the enzyme Lp-PLA₂. Thus, WO 99/24420, WO 00/10980, WO 00/66566, WO 00/66567 and WO 00/68208 (SmithKline Beecham plc) disclose a class of pyrimidone compounds. We have now found that the pyrimidone ring, optionally replaced by a pyridone ring, may be fused to a substituted benzo or pyrido ring to give compounds having good activity as inhibitors of the enzyme Lp-PLA₂. Accordingly, the present invention provides a compound of formula (I): 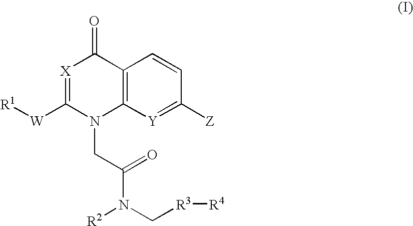
in which:
R¹ is an aryl group, optionally substituted by 1, 2, 3 or 4 substituents which may be the same or different selected from C₍₁₋₆₎alkyl, C₍₁₋₆₎alkoxy, C₍₁₋₆₎alkylthio, hydroxy, halogen, CN, mono to perfluoro-C₍₁₋₄₎alkyl, mono to perfluoro-C₍₁₋₄₎alkoxyaryl, and arylC₍₁₋₄₎alkyl;
R² is hydrogen, C₍₁₋₆₎alkyl which may be unsubstituted or substituted by 1, 2 or 3 substituents selected from hydroxy, halogen, OR⁵, COR⁵, carboxy, COOR⁵, CONR⁷R⁸, NR⁷R⁸, NR⁵COR⁶, mono- or di-(hydroxyC₍₁₋₆₎alkyl)amino and N-hydroxyC₍₁₋₆₎alkyl-N-C₍₁₋₆₎alkylamino; or
R² is Het-C₍₀₋₄₎alkyl in which Het is a 5- to 7-membered heterocyclyl ring comprising N and optionally O or S, and in which N may be substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl;
R³ is an aryl or a heteroaryl ring optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁵, carboxy, COOR⁵, NR⁵COR⁶, CONR⁷R⁸, SO₂NR⁷R⁸, NR⁵So₂R⁶, NR⁷R⁸, mono to perfluoro-C₍₁₋₄₎alkyl and mono to perfluoro-C₍₁₋₄₎alkoxy;
R⁴ is an aryl or a heteroaryl ring which is further optionally substituted by 1, 2, 3 or 4 substituents which may be the same or different selected from C₍₁₋₆₎alkyl, C₍₁₋₆₎alkoxy, C₍₁₋₆₎alkylthio, C₍₁₋₆₎alkylsulfonyl, arylC₍₁₋₆₎alkoxy, hydroxy, halogen, CN, COR⁵, carboxy, COOR⁵, CONR⁷R⁸, NR⁵COR⁶, SO₂NR⁷R⁸, NR⁵SO₂R⁶, NR⁷R⁸, mono to perfluoro-C₍₁₋₄₎alkyl and mono to perfluoro-C₍₁₋₄₎alkoxy, or C₍₅₋₁₀₎alkyl;
W is a C₍₂₋₄₎alkylene group, optionally substituted by 1, 2 or 3 substituents selected from methyl and ethyl, CH═CH, (CH₂)ₙS or (CH₂)ₙO where n is 1, 2 or 3;
X and Y are independently CH or N;
Z is NO₂, NR⁵R⁹, OR⁹, SR⁹, SOR⁹, SO₂R⁹ or R¹⁰;
R⁵ and R⁶ are independently hydrogen or C₍₁₋₁₂₎alkyl, for instance C₍₁₋₄₎alkyl (e.g. methyl, ethyl or t-butyl);
R⁷ and R⁸ which may be the same or different is each selected from hydrogen, or C₍₁₋₁₂₎alkyl, or R⁷ and R⁸ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl, e.g benzyl, for instance morpholine or piperazine;
R⁹ is hydrogen or C₍₁₋₆₎alkyl optionally substituted by 1, 2 or 3 substituents which may be the same or different selected from hydroxy, halogen, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin4-yl, pyrrolidin-3-yl; and
R¹⁰ is C₍₁₋₆₎alkyl optionally substituted by 1, 2 or 3 substituents which may be the same or different selected from hydroxy, halogen, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl; or
R¹⁰ is a 5- to 7-membered heterocyclic ring optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl;
with the provisos that Z is not amino and that the compound of formula (I) is not:
N-(2-diethylaminoethyl)-2-(2-(2-(2,3fluorophenyl)-ethyl)-7-trifluoromethyl-4-oxo-4H-quinazolin-1-yl)-N-(4′-trifluoromethyl-biphenyl-4-ylmethyl)acetamide;
N-(2-diethylaminoethyl)-2-(2-(2-(2,3-difluorophenyl)-ethyl)-7-methyl-4-oxo-4H-quinazolin-1-yl)-N-(4′-trifluoromethyl-biphenyl-4-ylmethyl)acetamide;
N-(1-ethylpiperidin-4-yl)-2-[2-(2-(2,3-difluorophenyl)ethyl)-7-methyl4-oxo-4H-[1,8]naphthyridin-1-yl]-N-(4′-trifluoromethylbiphenyl-4-ylmethyl)acetamide;
N-(2-diethylaminoethyl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide;
N-(1-methylpiperidin-4-yl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide;
N-(1-isopropylpiperidin-4-yl)-2-[2-(2-(2,3-difluorophenyl)ethyl)-7-methyl4-oxo4H-[1,8]naphthyridin-1-yl]-N-(4′-trifluoromethylbiphenyl-4-ylmethyl)acetamide; or
N-(1-(2-methoxyethyl)piperidin-4-yl)-2-[2-(2-(2,3-difluorophenyl)ethyl)-7-methyl-4-oxo-4H-[1,8]naphthyridin-1-yl]-N-(4′-trifluoromethylbiphenyl4-ylmethyl)acetamide.

In one aspect the aryl group of R¹ may be phenyl or naphthyl. Preferably, R¹ is phenyl optionally substituted by halogen, C₍₁₋₆₎alkyl, trifluoromethyl, C₍₁₋₆₎alkoxy, preferably, from 1 to 3 fluoro, more preferably, 2,3-difluoro.

In another aspect R² may be hydrogen, methyl, ethyl, isopropyl, 2-(diethylamino)ethyl, 2-(piperidin-1-yl)ethyl, 2-(pyrrolidin-1-yl)ethyl, 1-(2-methoxyethyl)piperidin4-yl, 1-methylpiperidin-4yl, 1-ethyl-piperidin-4-yl or 1-ethyl-pyrrolidin-2-ylmethyl. Preferably R² is methyl, ethyl, isopropyl or 1-ethyl-piperidin-4-yl especially methyl or ethyl.

In another aspect R³ may be phenyl or pyridyl. Preferably, R³ is phenyl.

In another aspect R⁴ may be phenyl optionally substituted by halogen, or trifluoromethyl, preferably at the 4-position, or ethyl. Preferably, R⁴ is phenyl substituted by trifluoromethyl at the 4-position.

Preferably, R³ and R⁴ together form a 4-(phenyl)phenyl or a 2-(phenyl)pyridinyl substituent in which the remote phenyl ring may be optionally substituted by halogen or trifluoromethyl, preferably at the 4-position.

Preferably W is (CH₂)ₙS or CH₍₂₋₄₎alkylene e.g. C₍₂₋₃₎alkylene, most preferably W is (CH₂)₂ or CH₂S.

In another aspect X may be CH.

In another aspect Y may be CH.

In another aspect Z may be NO₂, OR⁹ or R¹⁰.

In another aspect Z may be:
NO₂;
NR⁵R⁹, OR⁹, SR⁹, SOR⁹ or SO₂R⁹ where R⁹ is as hereinbefore defined; or
R¹⁰ where R¹⁰ is C₍₂₋₆₎alkyl, or C₍₁₋₆₎alkyl substituted by 1, 2 or 3 substituents which may be the same or different selected from hydroxy, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin4-yl, pyrrolidin-3-yl, or R¹⁰ is a 5- to 7-membered heterocyclic ring optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl.

In another aspect Z may be:
NO₂;
SOR⁹, SO₂R⁹ or NR⁵R⁹ where R⁹ is C₍₁₋₆₎alkyl;
OR⁹, SR⁹, SOR⁹, SO₂R⁹ or NR⁵R⁹ where R⁹ is hydrogen or mono to perfluoro-C₍₁₋₆₎alkyl;
OR⁹, SR⁹, SOR⁹, SO₂R⁹ or NR⁵R⁹ where R⁹ is C₍₁₋₆₎alkyl substituted by 1, 2 or 3 substituents which may be the same or different selected from hydroxy, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl; or
R¹⁰ where R¹⁰ is C₍₁₋₆₎alkyl substituted by 1, 2 or 3 substituents which may be the same or different selected from hydroxy, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl, or R¹⁰ is a 5- to 7-membered heterocyclic ring optionally substituted by COR⁵, COOR⁵, CONR⁷R⁸, or C₍₁₋₆₎alkyl optionally substituted by 1, 2 or 3 substituents selected from hydroxy, halogen, OR⁵, COR⁵, carboxy, COOR⁵, CONR⁷R⁸ or NR⁷R⁸, for instance, piperidin-4-yl, pyrrolidin-3-yl; with the proviso that when X is CH, Z is not C₍₁₋₃₎alkoxyC₍₁₋₃₎alkyl.

In another aspect Z may be hydroxy, nitro, mono or di-N-C₍₁₋₆₎alkylaminoC₍₁₋₆₎alkyl, mono or di-N-C₍₁₋₆₎alkylaminoC₍₁₋₆₎alkoxy, carboxyC₍₁₋₆₎alkoxy or an ester thereof, or arylC₍₁₋₆₎alkoxy, arylC₍₁₋₆₎alkyl, heteroarylC₍₁₋₆₎alkoxy, heteroarylC₍₁₋₆₎alkyl, 5- to 7-membered heterocyclylC₍₁₋₆₎alkoxy optionally substituted by C₍₁₋₆₎alkyl, or 5- to 7-membered heterocyclylC₍₁₋₆₎alkyl optionally substituted by C₍₁₋₆₎alkyl.

When Z includes an aryl, heteroaryl or heterocyclyl ring, said ring is preferably selected from benzyl, pyridinyl isoxazolyl, piperidinyl, pyrrolidinyl and morpholino, particularly piperidinyl and morpholino.

In another aspect Z may be 3-(dimethylamino)propyl, 3-(dimethylamino)propoxy, nitro, 2-(dimethylamino)ethoxy, 2-(diethylamino)ethoxy, 2-(piperidin-1-yl)ethoxy, 3-(piperidin-1-yl)propoxy, OCH₂CO₂^{t}Bu, (pyridin-2-yl)methoxy, (5-methylisoxazol-3-yl)methoxy, (1-methylpyrrolidin-2-yl)methoxy, benzyloxy, hydroxy, OCH₂CO₂H, dimethylaminomethyl, diethylaminomethyl, (pyrrolidin-1-yl)methyl, (piperidin-1-yl) methyl, 2-dimethylaminoethyl, 2-diethylaminoethyl, 2-(pyrrolidin-1-yl)ethyl, 3-diethylaminopropyl, 3-(pyrrolidin-1-yl)propyl, 3-(piperidin-1-yl)propyl or 3-(4-morpholino)propyl, particularly 3-(piperidin-1-yl)propyl or 3-(4-morpholino)propyl.

It will be appreciated that compounds of the present invention may comprise one or more chiral centres so that one or more stereoisomers may be formed.

The present invention encompasses all stereoisomers of the compounds of formula (I) including geometric isomers and optical isomers (eg. diastereoisomers and enantiomers) whether as individual stereoisomers isolated such as to be substantially free of the other stereoisomers (ie. pure) or as mixtures thereof including racemic modifications. An individual stereoisomer isolated such as to be substantially free of other stereoisomer (ie. pure) will preferably be isolated such that less than 10% preferably less than 1% especially less than 0.1% of the other stereoisomers is present.

Certain compounds of formula (I) may exist in one of several tautomeric forms. It will be understood that the present invention encompasses all tautomers of the compounds of formula (I) whether as individual tautomers or as mixtures thereof.

It will be appreciated that in some instances, compounds of the present invention may include a basic function such as an amino group as a substituent. Such basic functions may be used to form acid addition salts, in particular pharmaceutically acceptable salts. Pharmaceutically acceptable salts include those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, taurocholic acid, benzenesulfonic, p-toluenesulfonic, hydrochloric, hydrobromic, sulfuric, cyclohexylsulfamic, phosphoric and nitric acids.

It will be appreciated that in some instances, compounds of the present invention may include a carboxy group as a substituent. Such carboxy groups may be used to form salts, in particular pharmaceutically acceptable salts. Pharmaceutically acceptable salts include those described by Berge, Bighley, and Monkhouse, J. Pharm. Sci., 1977, 66, 1-19. Preferred salts include alkali metal salts such as the sodium and potassium salts.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the term “aryl” refers to, unless otherwise defined, a mono- or bicyclic aromatic ring system containing up to 10 carbon atoms in the ring system, for instance phenyl or naphthyl.

When used herein, the term “heteroaryl” refers to a mono- or bicyclic heteroaromatic ring system comprising up to four, preferably 1 or 2, heteroatoms each selected from oxygen, nitrogen and sulphur. Each ring may have from 4 to 7, preferably 5 or 6, ring atoms. A bicyclic heteroaromatic ring system may include a carbocyclic ring.

When used herein, the term “heterocyclyl” refers to, unless otherwise defined, a single or fused non-aromatic ring comprising up to four heteroatoms in the ring selected from oxygen, nitrogen and sulphur and optionally substituted with up to three substituents.

Suitably the heterocyclic ring comprises from 5 to 7, preferably 5 or 6, ring atoms. A fused heterocyclic ring system may include carbocyclic rings and need only include one heterocyclic ring.

When used herein, the terms “halogen” and “halo” include fluorine, chlorine, bromine and iodine and fluoro, chloro, bromo and iodo, respectively.

It is to be understood that the present invention covers all combinations of substituent groups referred to hereinabove.

Representative compounds of formula (I) are:
N-Methyl-2-(2-(2-(2,3-difluorophenyl)ethyl)-7-(3-dimethylaminoprop-1-yl)-4-oxo-4H-quinolin-1-yl)-N-(4-(4-trifluoromethylphenyl)benzyl)acetamide bitartrate;
2-(2-(2-(2,3-Difluorophenyl)ethyl)-4-oxo-7-(3-(dimethylamino)propoxy)-4H-quinolin-1-yl)-N-methyl-N-(4-(4-trifluoromethylphenyl)benzyl)acetamide bitartrate;
N-Methyl-2-(2-(2-(2,3-difluorophenyl)ethyl)-7-nitro4-oxo-4H-quinolin-1-yl)-N-(4-(4-trifluoromethylphenyl)benzyl) acetamide;
2-(2-(2-(2,3-Difluorophenyl)ethyl)-7-(2-dimethylaminoethoxy)-4-oxo-4H-quinolin-1-yl)-N-methyl-N-(4-(4-trifluoromethylphenyl)benzyl)acetamide bitartrate;
2-(7-(2-Diethylaminoethoxy)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(2-(2-(2,3-Difluorophenyl)ethyl)-4-oxo-7-(2-(piperidin-1-yl)ethoxy)-4H-quinolin-1-yl)-N-methyl-N-(4-(4-trifluoromethylphenyl)benzyl)acetamide bitartrate;
2-(2-(2-(2,3-Difluorophenyl)ethyl)-4-oxo-7-(3-(piperidin-1-yl)propoxy)-4H-quinolin-1-yl)-N-methyl-N-(4-(4-trifluoromethylphenyl)benzyl)acetamide bitartrate;
tert. Butyl 2-(2-(2-(2,3-difluorophenyl)ethyl))-1-(N-(4-(4-trifluoromethylphenyl)benzyl)-N-methyl-aminocarbonylmethyl)-4-oxo-4H-quinolin-7-yloxy)acetate;
2-(2-(2-(2,3-Difluorophenyl)ethyl)-4-oxo-7-(pyridin-2-ylmethoxy)-4H-quinolin-1-yl)-N-methyl-N-(4-(4-trifluoromethylphenyl)benzyl)acetamide hydrochloride;
2-(2-(2-(2,3-Difluorophenyl)ethyl)-7-(5-methylisoxazol-3-ylmethoxy)4-oxo-4H-quinolin-1-yl)-N-methyl-N-(4-(4-trifluoromethylphenyl)benzyl)acetamide;
2-(2-(2-(2,3-Difluorophenyl)ethyl)-7-(1-methylpyrrolidin-2-ylmethoxy)-4-oxo-4H-quinolin-1-yl)-N-methyl-N-(4-(4-trifluoromethylphenyl)benzyl)acetamide bitartrate;
2-(7-Benzyloxy-2-(2-(2,3-difluorophenyl)ethyl)-4-oxo-4-quinolin-1-yl)-N-methyl-N-(4-(4-trifluoromethylphenyl)benzyl)acetamide;
2-(7-Benzyloxy-2-(2-(2,3-difluorophenyl)ethyl)4-oxo4-quinolin-1-yl)-N-(1-ethyl-piperidin-4-yl)-N-(4-(4-trifluoromethylphenyl)benzyl)acetamide bitartrate;
2-(2-(2-(2,3-Difluorophenyl)ethyl)-7-hydroxy4-oxo-4H-quinolin-1-yl)-N-methyl-N-(4-(4-trifluoromethylphenyl)benzyl)acetamide;
2-(2-(2-(2,3-Difluorophenyl)ethyl)-7-hydroxy4-oxo-4H-quinolin-1-yl)-N-(1-ethyl-piperidin-4-yl)-N-(4-(4-trifluoromethylphenyl)benzyl)acetamide bitartrate;
2-(2-(2-(2,3-Difluorophenyl)ethyl))-1-(N-(4-(4-trifluoromethylphenyl)benzyl)-N-methyl-aminocarbonylmethyl)4-oxo-4H-quinolin-7-yloxy)acetic acid
2-(7-(Dimethylaminomethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(Diethylaminomethyl)-2-(2-(2,3-difluorophenylethyl)-4-oxo-4H-quinolin-1-yl)-N-methyl-N-(4-(4-trifluoromethylphenyl)benzyl)acetamide bitartrate;
2-(7-(Diethylaminomethyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-(Diethylaminomethyl)-2-(2-(2,3-difluorophenyl)ethyl)-4-oxo-4H-quinolin-1-yl)-N-isopropyl-N-(4-(4-trifluoromethylphenyl)benzyl)acetamide bitartrate;
2-(7-((Pyrrolidin-1-yl)methyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-((Piperidin-1-yl)methyl)-2-(2-(2,3-difluorophenyl)ethyl)4-oxo-4H-quinolin-1-yl)-N-ethyl-N-(4-(4-trifluoromethylphenyl)benzyl)acetamide bitartrate;
2-(7-(2-Dimethylaminoethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(2-Diethylaminoethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(2-(Pyrrolidin-1-yl)ethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(3-Diethylaminopropyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(3-(Pyrrolidin-1-yl)propyl)-2-(2-(2,3-difluorophenyl)ethyl)-4-oxo4H-quinolin-1yl)-N-methyl-N-(4-(4-trifluoromethylphenyl)benzyl)acetamide bitartrate;
2-(7-(3-(Piperidin-1-yl)propyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-(3-(Piperidin-1-yl)propyl)-2-(2-(2,3-difluorophenyl)ethyl)-4-oxo-4H-quinolin-1yl)-N-isopropyl-N-(4-(4-trifluoromethylphenyl)benzyl)acetamide bitartrate;
2-(7-(Diethylaminomethyl)-2-(2,3-difluorobenzylthio)-4-oxo-4H-quinolin-1-yl)-N-methyl-N-(4-(4-trifluoromethylphenyl)benzyl)acetamide bitartrate; and
2-(7-(3-(4-Morpholino)propyl)-2-(2,3-difluorobenzylthio)-4-oxo-4H-quinolin-1-yl)-N-methyl-N-(4-(4-trifluoromethylphenyl)benzyl)acetamide hydrochloride.

Preferred compounds are:
2-(7-(3-(Piperidin-1-yl)propyl)-2-(2-(2,3-difluorophenyl)ethyl)-4-oxo-4H-quinolin-1-yl)-N-ethyl-N-(4-(4-trifluoromethylphenyl)benzyl)acetamide bitartrate; and
2-(7-(3-(4-Morpholino)propyl)-2-(2,3-difluorobenzylthio)-4-oxo-4H-quinolin-1-yl)-N-methyl-N-(4-(4-trifluoromethylphenyl)benzyl)acetamide hydrochloride.

Preferred salts are the bitartrate and hydrochloride salts.

Since the compounds of the present invention, in particular compounds of formula (I), are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are re-crystallised from organic solvents, solvent of crystallisation may be present in the crystalline product. This invention includes within its scope such solvates. Similarly, some of the compounds of this invention may be crystallised or re-crystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

Compounds of the present invention are inhibitors of the enzyme lipoprotein associated phospholipase A₂ (Lp-PLA₂) and as such are expected to be of use in therapy, in particular in the treatment of atherosclerosis. In a further aspect therefore the present invention provides a compound of formula (I) for use in therapy.

The compounds of formula (I) are inhibitors of lysophosphatidylcholine production by Lp-PLA₂ and may therefore also have a general application in any disorder that involves endothelial dysfunction, for example atherosclerosis, diabetes, hypertension, angina pectoris and after ischaemia and reperfusion. In addition, compounds of formula (I) may have a general application in any disorder that involves lipid oxidation in conjunction with enzyme activity, for example in addition to conditions such as atherosclerosis and diabetes, other conditions such as rheumatoid arthritis, stroke, inflammatory conditions of the brain such as Alzheimer's Disease, myocardial infarction, ischaemia, reperfusion injury, sepsis, and acute and chronic inflammation.

Further applications include any disorder that involves activated monocytes, macrophages or lymphocytes, as all of these cell types express Lp-PLA₂. Examples of such disorders include psoriasis.

Accordingly, in a further aspect, the present invention provides for a method of treating a disease state associated with activity of the enzyme Lp-PLA₂ which method involves treating a patient in need thereof with a therapeutically effective amount of an inhibitor of the enzyme. The disease state may be associated with the increased involvement of monocytes, macrophages or lymphocytes; with the formation of lysophosphatidylcholine and oxidised free fatty acids; with lipid oxidation in conjunction with Lp PLA₂ activity; or with endothelial dysfunction.

Compounds of the present invention may also be of use in treating the above mentioned disease states in combination with an anti-hyperlipidaemic, anti-atherosclerotic, anti-diabetic, anti-anginal, anti-inflammatory, or anti-hypertension agent or an agent for lowering Lp(a). Examples of the above include cholesterol synthesis inhibitors such as statins, anti-oxidants such as probucol, insulin sensitisers, calcium channel antagonists, and anti-inflammatory drugs such as NSAIDs. Examples of agents for lowering Lp(a) include the aminophosphonates described in WO 97/02037, WO 98/28310, WO 98/28311 and WO 98/28312 (Symphar SA and SmithKline Beecham).

A preferred combination therapy will be the use of a compound of the present invention and a statin. The statins are a well known class of cholesterol lowering agents and include atorvastatin, simvarstatin, pravastatin, cerivastatin, fluvastatin, lovastatin and ZD 4522 (also referred to as S-4522, rosuvastatin, Astra Zeneca). The two agents may be administered at substantially the same time or at different times, according to the discretion of the physician.

A further preferred combination therapy will be the use of a compound of the present invention and an anti-diabetic agent or an insulin sensitiser, as coronary heart disease is a major cause of death for diabetics. Within this class, preferred compounds for use with a compound of the present invention include the PPARgamma activators, for instance GI262570 (GlaxoSmithKIine) and the glitazone class of compounds such as rosiglitazone (Avandia, GlaxoSmithkline), troglitazone and pioglitazone.

In therapeutic use, the compounds of the present invention are usually administered in a standard pharmaceutical composition. The present invention therefore provides, in a further aspect, a pharmaceutical composition comprising a compound of formula (I) and a pharmaceutically acceptable carrier, optionally with one or more other therapeutic compounds such as a statin or an anti-diabetic.

Suitable pharmaceutical compositions include those which are adapted for oral or parenteral administration or as a suppository, particularly for oral administration.

Compounds of formula (I) which are active when given orally can be formulated as liquids, for example syrups, suspensions or emulsions, tablets, capsules and lozenges. A liquid formulation will generally consist of a suspension or solution of the compound or pharmaceutically acceptable salt in a suitable liquid carrier(s) for example, ethanol, glycerine, non-aqueous solvent, for example polyethylene glycol, oils, or water with a suspending agent, preservative, flavouring or colouring agent. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule. Typical parenteral compositions consist of a solution or suspension of the compound of formula (I) in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. A typical suppository formulation comprises a compound of formula (I) which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule. Each dosage unit for oral administration contains preferably from 1 to 500 mg (and for parenteral administration contains preferably from 0.1 to 25 mg) of a compound of the formula (I). The daily dosage regimen for an adult patient may be, for example, an oral dose of between 1 mg and 1000 mg, preferably between 1 mg and 500 mg, or an intravenous, subcutaneous, or intramuscular dose of between 0.1 mg and 100 mg, preferably between 0.1 mg and 25 mg, of the compound of the formula (I), the compound being administered 1 to 4 times per day. Suitably the compounds will be administered for a period of continuous therapy, for example for a week or more.

According to a first process A, a compound of formula (I) may be prepared by reacting an acid compound of formula (II): 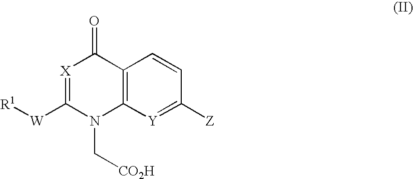
in which W, X, Y, Z and R¹ are as hereinbefore defined,
with an amine compound of formula (III):
   R⁴—R³—CH₂NHR² (III)
   in which R², R³ and R⁴ are as hereinbefore defined; under amide forming conditions.

Suitable amide forming conditions are well known in the art and include treating the acid of formula (II) with the amine of formula (III) in the presence of a coupling agent such as 1-(3-dimethyl-aminopropyl)-3-ethylcarbodiimide (DEC) or O-(7-azabenzotriazol-1-yl)-N,N′,N′-tetramethyluronium hexafluorophosphate (HATU) in an aprotic solvent such as dichloromethane or dimethylformamide (DMF).

A compound of formula (II) may be readily prepared from a corresponding ester of formula (IV): 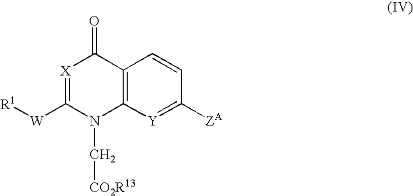
in which W, X, Y and R¹ are as hereinbefore defined, Z^{A} is Z as hereinbefore defined or a group convertible to Z, and R¹³ is C₍₁₋₆₎alkyl, for example ethyl or t-butyl, by treating with a de-esterifying agent, for instance, for t-butyl, trifluoroacetic acid.

It will be appreciated that removal of R¹³ may be carried out as a separate step, so that an acid of formula (II), or a salt thereof, for example the sodium salt, is isolated or, alternatively, that the acid of formula (II), or a salt thereof, is formed from the intermediate ester (IV) as a preliminary first step, prior to reaction with an amine of formula (III).

Thus, according to another process B a compound of formula (I) can be prepared by (a) treating a compound of formula (IV) with a deesterifying agent to form a compound of formula (II) or a salt thereof; and (b) treating said compound of formula (II) or salt thereof with an amine compound of formula (III) under amide forming conditions.

When X is N and Y is CH, the ester of formula (IV) may be readily prepared by reacting an amidine of formula (V): 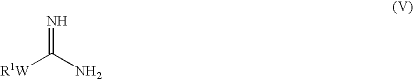
in which R¹ and W are as hereinbefore defined,
preferably as a salt thereof, for instance the hydrochloride salt,
with a compound of formula (VI): 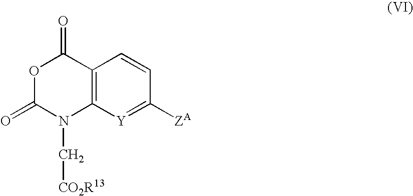
   in which Z^{A} is Z as hereinbefore defined or a group convertible to Z, and R¹³ is as hereinbefore defined, for example ethyl, under standard pyrimidone ring forming conditions, in the presence of a base such as pyridine, to give an intermediate ester (IV) which can then be converted into a compound of formula (II), for instance by treatment with aqueous sodium hydroxide.

Alternatively, when X is N and Y is CH, the pyrimidone ring may be formed by reacting a compound of formula (VII): 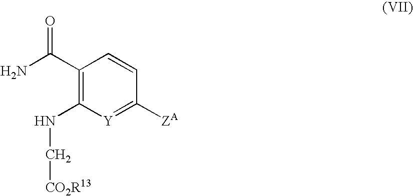
in which Z^{A} is Z as hereinbefore defined or a group convertible to Z, and R¹³ is as hereinbefore defined, for example ethyl, with an acyl chloride compound of the formula (VIII): 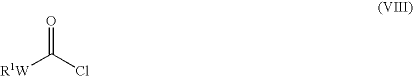
in which R¹ and W are as hereinbefore defined;
under standard pyrimidone ring forming conditions, in a solvent such as benzene, or via a two step procedure by treatment with pyridine, followed by a suitable base e.g. NaH in DMF, followed by treatment of the intermediate so formed with an acid e.g. p-toluene sulfonic acid in refluxing toluene; to give an intermediate ester (IV) which can then be converted into a compound of formula (II), for instance by treatment with aqueous sodium hydroxide.

When X and Y are CH, the overall synthesis of compounds of formula (I) is illustrated in the following scheme:

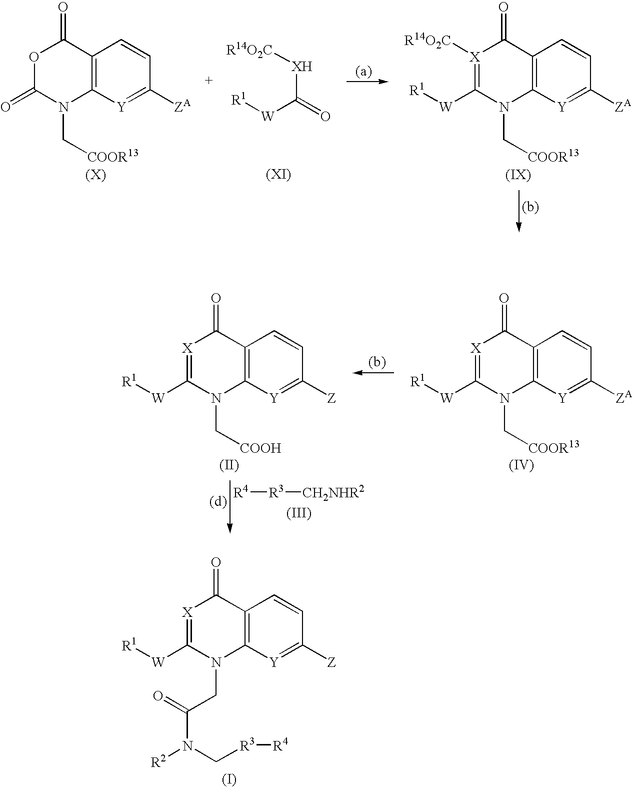

Referring to the scheme, the key intermediate (IV) may be synthesised by removing the 3-ester group from intermediate (IX) wherein R¹⁴ is C₍₁₋₆₎alkyl, for example by heating in diphenyl ether where R¹⁴ is ^{t}Bu (step b). Intermediate (m) is formed from the 2,6-dioxo-1,3-oxazine (X) and ester (XI) by treatment with a base, for example 1,8-diazabicyclo[5.4.0]undec-7-ene in tetrahydrofuran or NaH in DMF.

Alternatively where W is S and X and Y are CH, the synthesis of intermediate (IV) is illustrated in the following scheme:

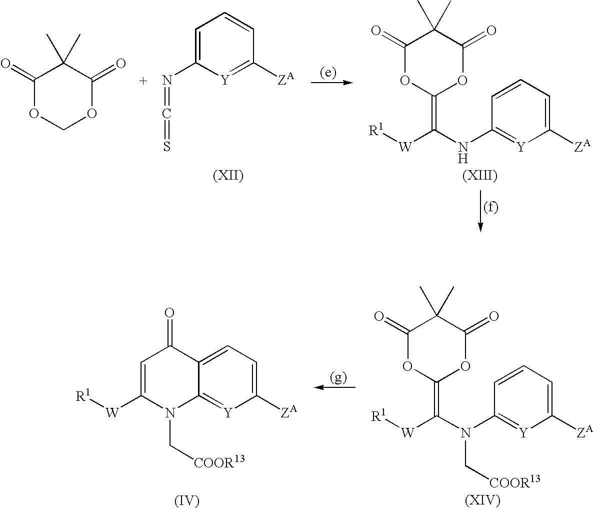

Referrring to the scheme, the key intermediate (IV) may be synthesised by acid catalysed cyclisation of intermediate (XIV), for example by heating with trifluoromethanesulfonic acid in dichloromethane. Intermediate (XIV) is formed by alkylation of intermediate (XIII) with a compound of formula (XV):
L—CH₂—COOR¹³ (XV)
in which L is a leaving group, for example chloro or bromo, in the presence of a base such as potassium tert-butoxide, in a solvent such as N-methylpyrrolidone. Intermediate (XIII) is formed by reaction of Meldrum's acid with a compound of formula (XII) in the presence of a base such as N,N-diispropylethylamine in a solvent such as N-methylpyrrolidone, immediately followed by treatment with an alkylating agent for formula (XVI):
L—R¹ (XVI)
for example 2,3-difluorobenzyl bromide.

Conversion of Z^{A} to Z typically arises if a protecting group, or a group which can take part in subsequent reactions such as coupling reactions, is needed during the above reactions or during the preparation of the reactants. The conversion of Z^{A} to Z may be carried out at different stages in the synthesis of the compounds of formula (I) depending on the nature of Z, including as a final step.

Z^{A} may be, for example, a protected hydroxy group. Suitable protecting groups are those well known in the art which may be removed under conventional conditions and without disrupting the remainder of the molecule. A comprehensive discussion of the ways in which groups may be protected and methods for cleaving the resulting protected derivatives is given in for example Protective Groups in Organic Chemistry, T. W. Greene and P. G. M. Wuts, (Wiley-Interscience, New York, 2nd edition, 1991). Particularly suitable hydroxy protecting groups include benzyl.

Thus, according to another process C, a compound of formula (I) may be prepared by subjecting a protected derivative of a compound of formula (I) to reaction to remove the protecting group or groups present, constituting a further aspect of the present invention.

Z^{A} may also be a group such as halo, for example chloro, bromo or iodo, which can be converted to Z at different stages in the synthesis of the compounds of formula (I), including as a final step using one of the general methods for functional group transformation described in the literature provided that the method chosen is compatible with the other functional groups in the molecule. Functional group transformations are well known in the art and are described in for instance Comprehensive Organic Functional Group Transformations, eds. A. R. Katritzky, O. Meth-Cohn and C. W. Rees (Elsevier Science Ltd., Oxford, 1995), Comprehensive Organic Chemistry, eds. D. Barton and W. D. Ollis (Pergamon Press, Oxford, 1979), and Comprehensive Organic Transformations, R. C. Larock (VCH Publishers Inc., New York, 1989). Some representative examples of transformations based on intermediates where Z^{A} is halo are shown in the following scheme (part structures shown):

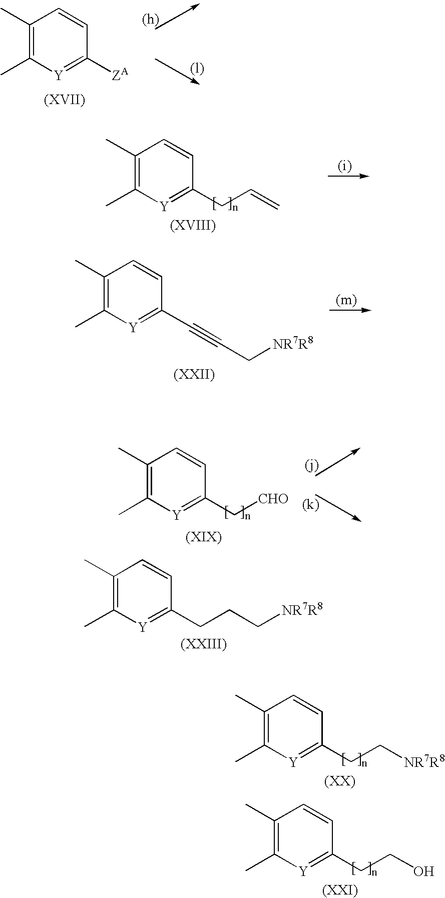

Thus an intermediate with part-structure (XVII), in which Z^{A} is bromo or iodo, can undergo palladium-catalysed coupling with vinyl stannanes to form (XVIII) where n=0, or with allyl stannanes to form (XVIII) where n=1 (step h). Oxidative cleavage of the terminal alkene group of (XVIII), for example by oxidation with osmium tetroxide followed by treatment with sodium periodate (step i), forms an aldehyde of part structure (XIX). For the case where n=2, coupling of (XVII) with allyl alcohol gives (XIX) directly. Such compounds, in which Z^{A} is (CH₂)ₙCHO, in turn represent versatile intermediates. For example, reductive amination with an amine of formula NHR⁷R⁸ and a reducing agent such as sodium triacetoxyborohydride forms (XX), in which Z is (CH₂)ₙ₊₁NR⁷R⁸; or reduction by standard means forms alcohols (XXI). In a further example, longer chain substituents can conveniently be formed by palladium-catalysed coupling of alkynes to (XVII), in which Z^{A} is bromo or iodo (step l), and subsequent reduction (step m).

Thus, according to another process D, a compound of formula (I) may be prepared from a compound of formula (I) in which Z^{A} is a group convertible to Z by functional group transformation, constituting another aspect of the present invention.

It will further be appreciated that compounds of formula (I) may also be prepared from other compounds of formula (I) using conventional interconversion procedures (process E), constituting yet a further aspect of the present invention.

The following Examples illustrate the invention.

### EXAMPLES

The structure and purity of the intermediates and examples was confirmed by 1H-NMR and (in nearly all cases) mass spectroscopy, even where not explicitly indicated below.

### Intermediate A1

4-Bromo-2-nitrobenzoic Acid.

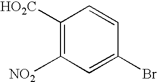

Potassium permanganate (29.6 g, 187 mmol) was added in portions over 8 h to a refluxing solution of 4-bromo-2-nitrotoluene (10.1 g, 46.75 mmol) in pyridine (80 ml) and water (70 ml). The suspension was filtered whilst hot and the resultant yellow solution was evaporated to about ¼ volume. Sodium hydroxide (2M, 20 ml) was added and the solution extracted with diethyl ether (to remove any 4-bromo-2-nitrotoluene that remained). The solution was acidified with hydrochloric acid (conc.) and the resultant solid collected and dried under reduced pressure to afford the title compound (5.72 g, 50%). ¹H NMR (d₆-DMSO) δ 7.79 (1H, d), 7.98 (1H, dd), 8.23 (1H, d).

### Intermediate A2

Ethyl 4-bromo-2-nitrobenzoate

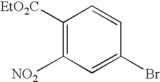

A solution of intermediate A1 (6.9 g, 28.05 mmol) and sulphuric acid (conc., 10 ml) in ethanol (80 ml) was heated to reflux for 18 h. After cooling the solvent was evaporated. The residue was dissolved in diethyl ether and washed with water, saturated sodium bicarbonate, dried (MgSO₄) and the solvent evaporated to afford the title compound (7.09 g, 92%). ¹H NMR (CDCl₃) δ 1.35 (3H, t), 4.39 (2H, q), 7.65 (1H, d), 7.78 (1H, dd), 8.00 (1H, d).

### Intermediate A3

Ethyl 2-amino-4-bromobenzoate.

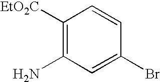

A mixture of intermediate A2 (7.09 g, 25.9 mmol) and iron (14.5 g, 259 mmol) in 10% aqueous ethanol (250 ml) was heated to reflux for 6 h. The mixture was filtered through celite and the resultant solution evaporated. The crude mixture was purified by chromatography (plug of silica gel) in dichloromethane to afford the title compound (4.53 g, 72%). ¹H NMR (CDCl₃) δ 1.37 (3H, t), 4.32 (2H, q), 5.77 (2H, br s), 6.73 (1H, dd), 6.83 (1H, d), 7.71 (1H, d).

### Intermediate A4

2-Amino-4-bromobenzoic Acid Hydrochloride.

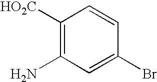

Sodium hydroxide (2M, 28 ml) was added to a stirred solution of intermediate A3 (4.53 g, 18.6 mmol) in dioxan (150 ml) and water (22 ml) and the resultant solution heated at 75° C. for 4 h. After cooling the solvent was evaporated and the residue suspended in water. The mixture was acidified (conc. HCl) and the resultant solid collected and dried to afford the title compound (4.29 g, 89%). ¹H NMR (d₆-DMSO) δ 6.64 (1H, dd), 6.97 (1H, d), 7.59 (1H, d).

### Intermediate A5

7-Bromo-1 H-benzo[d][1,3]oxazine-2,4-dione.

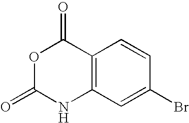

Phosgene (20% in toluene, 12.9 ml) was added dropwise to a stirred solution of intermediate A4 (3.15 g, 12.5 mmol) in dioxan (30 ml), stirring was continued for 18 h. The solvent was evaporated to afford the title compound (2.96 g, 98%). ¹H NMR (d₆-DMSO) δ 7.33 (1H, d), 7.42 (1H, dd), 7.83 (1H, d), 11.87 (1H, s); ¹³C NMR (d₆-DMSO) 1.09, 117.6, 126.4, 130.1, 130.7, 142.4, 146.7 and 159.2.

### Intermediate A6

Ethyl (7-bromo-2,4-dioxo-4 H-benzo[d][1,3]oxazin-1-yl)acetate.

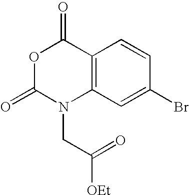

Ethyl bromoacetate (4 ml, 36.1 mmol) was added dropwise to a stirred solution of intermediate A5 (8.74 g, 36.1 mmol) and diisopropylethylamine (7.5 ml, 43.3 mmol) in N-methyl-2-pyrrolidinone (50 ml) at 0° C. Stirring was continued at room temperature for 18 h. The solution was diluted with water (50 ml) and the resultant solid collected and dried (MgSO₄) to afford the title compound (10 g, 85%). ¹H NMR (d₆-DMSO) δ 1.23 (3H, t), 4.20 (2H, q), 4.91 (2H, s), 7.41 (1H, dd), 7.81 (1H, d), 7.94 (1H, d).

### Intermediate A7

tert-Butyl 5-(2,3-difluorophenyl)-3-oxopentanoate

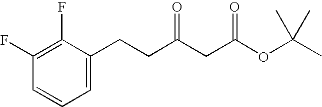

To an ice cooled stirring suspension of sodium hydride (1.96 g, 49.1 mmol, 60% dispersion in oil) in dry tetrahydrofuran (100 ml) was added dropwise under an argon atmosphere tert-butylacetoacetate (7.4 ml, 44.6 mmol). After a further 15 min, n-butyllithium(18.7 ml, 46.8 mmol, 2.5M in hexanes) was added dropwise maintaining the reaction temperature below 10° C. 2,3-Difluorobenzyl bromide (11.08 g, 53.5 mmol) was added dropwise 20 min later, then the mixture allowed to warm to ambient temperature. After a further 15 min the reaction mixture was poured onto a mixture of water (150 ml) and glacial acetic acid (10 ml), extracted 3 times with ethyl acetate and the combined extracts washed with saturated sodium hydrogen carbonate then brine, dried (MgSO₄) and evaporated to a yellow oil. Chromatography (fine silica, ethyl acetate-light petrol) gave the title compound as a yellow oil, yield 9.05 g (71%). 1H NMR (CDCl₃) δ 1.45 (9H, s), 2.84-2.91 (2H, m), 2.95-3.00 (2H, m), 3.35 (2H, s), 6.92-7.04 (3H, m).

### Intermediate A8

tert-butyl (7-Bromo-2-(2-(2,3-difluorophenyl)ethyl)-1-ethoxycarbonyl-4-oxo 4-H quinolin-1-yl)-3-carboxylate.

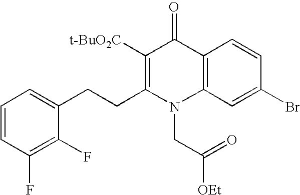

1,8-Diazabicyclo[5.4.0]undec-7-ene (9.1 ml, 61 mmol) was added dropwise to a stirred solution of intermediate A6 (10 g, 30.5 mmol) and intermediate A7 (9.52 g, 33.5 mmol) in tetrahydrofuran (100 ml) at 0° C. Stirring was continued at room temperature for 18 h. The solution was diluted with ethyl acetate, washed with saturated sodium bicarbonate, dried (MgSO₄) and the solvent evaporated. The residue was purified by chromatography (petrol/ethyl acetate) to afford the title compound (9.3 g, 55%). ¹H NMR (CDCl₃) δ 1.33 (3H, t), 1.62 (9H, s), 2.97 (2H, m), 3.07 (2H, m), 4.34 (2H, q), 4.94 (2H, br s), 7.00-7.12 (3H, m), 7.42 (1H, d), 7.49 (1H, dd), 8.29 (1H, d).

### Intermediate A9

Ethyl (7-bromo-2-(2-(2,3-difluorophenyl)ethyl)-4-oxo-4 H-quinolin-1-yl)acetate.

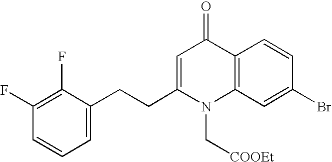

Intermediate A8 (9.3 g, 16.9 mmol) in diphenyl ether (30 ml) was heated to reflux for 15 min. After cooling petrol was added and the resultant solid collected by filtration. The crude product was purified by chromatography (silica gel, petrol/ethyl acetate) to afford the title compound (2.8 g, 37%). ¹H NMR (CDCl₃) δ 1.31 (3H, t), 2.91 (2H, m), 3.05 (2H, m), 4.32 (2H, q), 4.86 (2H, s), 6.25 (1H, s), 6.93-7.14 (3H, m), 7.42 (1H, d), 7.49 (1H, dd), 8.29 (1H, d); MS (APCI+) found (N+1)=450; C₂₁H₁₈⁷⁹BrNO₃ requires 449.

### Intermediate A10

Ethyl (2-(2-(2,3-difluorophenyl)ethyl)-7-(3-dimethylaminoprop-1-ynyl)-4-oxo-4 H-quinolin-1-yl)acetate.

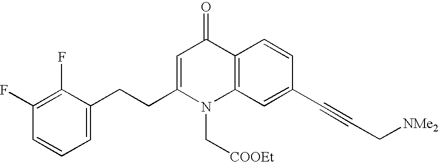

A mixture of Intermediate A9 (0.43 g, 0.96 mmol), 3-dimethylaminopropyne (0.41 ml, 3.8 mmol), bis(triphenylphosphine)palladium(II) chloride (0.07 g) and copper(I) iodide (0.04 g) in 1,2-dimethoxyethane (5 ml) and triethylamine (5 ml) was heated to 75° C. for 2 h. After cooling, the solvent was evaporated and the residue suspended in dichloromethane and washed with saturated sodium bicarbonate, dried (MgSO₄) and the solvent evaporated. The crude product was purified by chromatography (NH₃/MeOH/CH₂Cl₂) to afford the title compound (390 mg, 90%). ¹H NMR (CDCl₃) δ 1.30 (3H, t), 2.39 (6H, s), 2.92 (2H, m), 3.05 (2H, m), 3.49 (2H, s), 4.30 (2H, q), 4.88 (1H, s), 6.25 (1H, s), 6.93-7.10 (3H, m), 7.32 (1H, d), 7.42 (1H, dd), 8.35 (1H, d); MS (APCI+) found (M+1)=453; C₂₆H₂₆F₂F₃N₂ requires 452.

The following intermediates were prepared by the method of intermediate A10.

| No. | Precursor | Structure | Name |
|---|---|---|---|
| A90 | A9 | 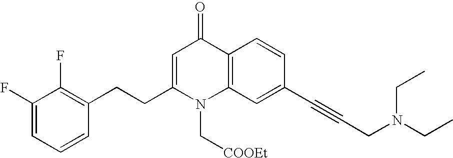 | Ethyl(7-(3-diethylaminoprop-1-ynyl)-2-(2-(2,3-di- fluorophenyl)-eth- yl)-4-oxo-4H-quinolin-1-yl)-ace- tate |
| A91 | A9 | 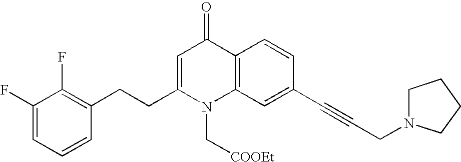 | Ethyl(7-(3-(pyrrolidin-1-yl)prop-1-ynyl)-2-(2-(2,3-di- fluorophenyl)-eth- yl)-4-oxo-4H-quinolin-1-yl)ace- tate |
| A92 | A9 | 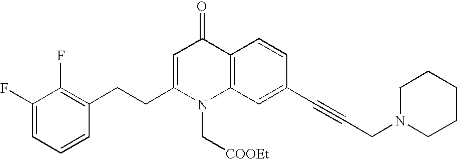 | Ethyl(7-(3-(piperidin-1-yl)prop-1-ynyl)-2-(2-(2,3-di- fluorophenyl)-eth- yl)-4-oxo-4H-quinolin-1-yl)-ace- tate |

### Intermediate A11

Ethyl (2-(2-(2,3-difluorophenyl)ethyl)-7-(3-dimethylaminopropyl)-4-oxo-4 H-quinolin-1-yl)acetate.

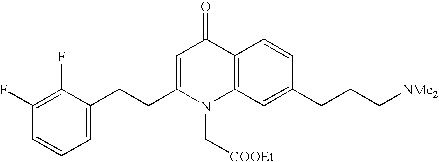

A mixture of intermediate A10 (0.39 g, 0.86 mmol) and Pd/C (40 mg) in ethanol was hydrogenated at room temperature and pressure until the reaction was complete by HPLC. The catalyst was filtered off and the solvent evaporated to afford the title compound (0.29 g, 73%). ¹H NMR (CDCl₃) δ 1.28 (3H, t), 1.83 (2H, m), 2.24 (6H, s), 2.31 (2H, t), 2.78 (2H, t), 2.92 (2H, m), 3.05 (2H, m), 4.28 (2H, q), 4.90 (2H, s), 6.25 (1H, s), 6.96-7.11 (4H, m), 7.23 (1H, dd), 8.34 (1H, d); MS (APCI+) found (M+1)=457; C₂₆H₃₀F₂N₂O₃ requires 456.

The following intermediates were prepared by the method of intermediate A11.

| No. | Precursor | Structure | Name |
|---|---|---|---|
| A100 | A90 | 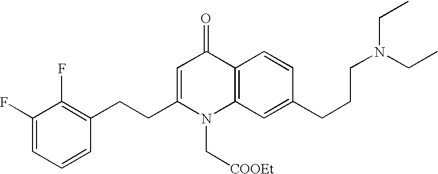 | Ethyl(7-(3-diethylaminopropyl)-2-(2-(2,3-di- fluorophenyl)ethyl)-4-oxo-4H-quino- lin-1-yl)acetate |
| A101 | A91 | 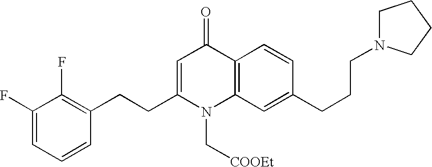 | Ethyl(7-(3-(pyrrolidin-1-yl)propyl)-2-(2-(2,3-di- fluorophenyl)ethyl)-4-oxo-4H-quino- lin-1-yl)acetate |
| A102 | A92 | 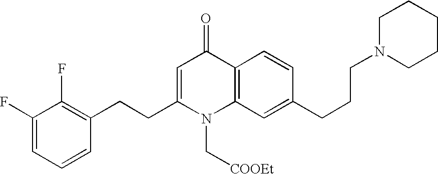 | Ethyl(7-(3-(piperidin-1-yl)propyl)-2-(2-(2,3-di- fluorophenyl)ethyl)-4-oxo-4H-quino- lin-1-yl)acetate |

### Intermediate A12

5-((2,3-difluorobenzylthio)-(3-iodophenylamino)methylene)-2,2-dimethyl-1,3-dioxane-4,6-dione.

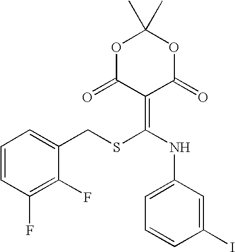

To a solution of Meldrum's acid (13.4 g, 93 mmol) in N-methylpyrrolidinone (50 ml), cooled to 15° C., was added N,N-diisopropylethylamine (15 ml, 86 mmol). The mixture was stirred at 15° C. for 1 h, then a solution of 3-iodophenylisothiocyanate (20.9 g, 80 mmol) in N-methylpyrrolidinone (90 ml) was added. The mixture was stirred at room temperature for 16 h, then cooled to 10° C. 2,3-Difluorobenzyl bromide (16.5 g, 80 mmol) was added over 20 minutes. The reaction mixture was stirred at room temperature for 3 h, then poured into a mixture of EtOAc and water. The aqueous phase was extracted with EtOAc and the combined organic phases washed twice with water, then brine, dried (Na₂SO₄) and evaporated. The residual oil was stirred with Et₂O, the resulting solid was collected by filtration to give the title compound (36 g). ¹H NMR (CDCl₃) δ 1.77 (6H, s), 4.07 (2H, s), 6.9-7.2 (4H, m), 7.2-7.3 (1H, br.), 7.6-7.75 (2H, br.), 12.8 (1H, s); MS (APCI−) found (M−1)=530; C₂₀H₁₆F₂INO₄S requires 531.

### Intermediate A13

Ethyl (7-iodo-2-(2,3-difluorobenzylthio)-4-oxo-4H-quinolin-1-yl)acetate.

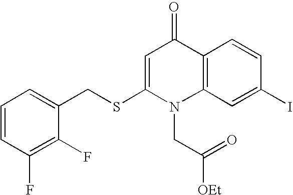

To a solution intermediate A12 (20 g, 38 mmol) in NMP (50 ml) was added potassium tert-butoxide (4.54 g, 40 mmol). The mixture was stirred at room temperature for 1 h, then ethyl bromoacetate (4.6 ml, 40 mmol) was added. The mixture was stirred at 70° C. for 8 h. EtOAc was added to the cooled reaction mixture which was washed with water, then brine, and evaporated. The residual oil was stirred with Et₂O, the resulting solid was collected by filtration to give a solid (13.6 g). To a solution of this solid (9.6 g) in CH₂Cl₂ (80 ml) at reflux was added trifluoromethanesulfonic acid (1.72 ml) portionwise over 90 minutes. After a further 2 h at reflux, the reaction mixture was cooled and poured into a mixture of saturated sodium bicarbonate and CH₂Cl₂. The organic phase was washed with water, dried (Na₂SO₄) and evaporated. Chromatography (silica gel, MeOH/CH₂Cl₂) gave ethyl 2-(2-(2,3-difluorophenyl)ethyl)-5-iodo-4-oxo-4H-quinolin-1-yl)acetate (2.05 g) as the earlier eluting isomer followed by ethyl 2-(2,3-difluorobenzylthio)-7-iodo-4-oxo-4H-quinolin-1-yl)acetate (title compound) (1.1 g). ¹H NMR (CDCl₃) δ 1.31 (3H, t), 4.28 (2H, s), 4.30 (2H, q), 5.08 (2H, br s), 6.40 (1H, s), 7.02-7.19 (3H, m), 7.56 (1H, s), 7.69 (1H, d), 8.08 (1H, d); MS (APCI+) found (M+1)=516; C₂₀H₁₆F₂INO₃S requires 515.

### Intermediate A110

Ethyl (7-vinyl-2-(2-(2,3-difluorophenyl)ethyl)-4-oxo-4H-quinolin-1-yl)acetate.

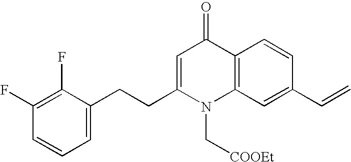

A mixture of intermediate A9 (0.6 g, 1.33 mmol), tributyl(vinyl)tin (1.0 ml, 3.42 mmol) and bis(triphenylphosphine)palladium(II) chloride (0.1 g) in NMP (8 ml) was stirred at 100° C. for 1 h. After cooling the mixture was diluted with ethyl acetate, washed with water then brine, dried (MgSO₄) and evaporated. Chromatography (silica gel, MeOH/CH₂Cl₂), then crystallisation (EtOAc) gave the title compound as a light grey solid (0.357 g, 67%). ¹H NMR (CDCl₃) δ 1.29 (3H, t), 2.92-2.97 (2H, m), 3.03-3.09 (2H, m), 4.30 (2H, q), 4.91 (2H, s), 5.45 (1H, d), 5.90 (1H, d), 6.26 (1H, s), 6.80 (1H, dd), 6.96-7.11 (3H, m), 7.18 (1H, s), 7.50 (1H, d), 8.38 (1H, d); MS (APCI+) found (M+1)=398; C₂₃H₂₁F₂NO₃ requires 397.

The following intermediates were prepared by the method of intermediate A110.

| No. | Precursor | Structure | Name |
|---|---|---|---|
| A111 | A9 | 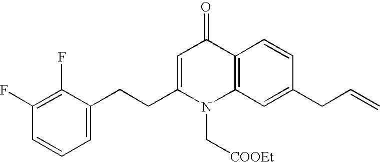 | Ethyl(7-allyl-2-(2-(2,3-di- fluorophenyl)ethyl)-4-oxo-4H-quino- lin-1-yl)acetate |
| A112 | A13 | 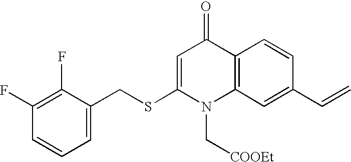 | Ethyl(2-(2,3-difluorobenzylthio)-7-vi- nyl-4-oxo-4H-quinolin-1-yl)acetate |

### Intermediate A120

Ethyl (7-formyl-2-(2-(2,3-difluorophenyl)ethyl)-4-oxo-4H-quinolin-1-yl)acetate.

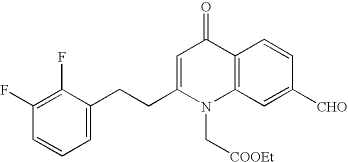

To a solution of intermediate A110 (0.549 g, 1.38 mmol) in a mixture of acetone, water and tert-butanol (30 ml, 4:2:1 ratio) was added 4-methylmorpholine N-oxide (0.336 g, 2.87 mmol) and osmium tetroxide (2.5 wt % solution in tert-butanol, 0.34 ml). The reaction mixture was stirred at room temperature for 5 h. A solution of sodium metabisulfite (10 wt % in water, 17 ml) was added and the mixture stirred for 1 h. Water (100 ml) and a mixture of CH₂Cl₂ and MeOH (150 ml, 9:1) were added. The aqueous layer was extracted with three further portions of CH₂Cl₂ and MeOH (9:1). The organic extracts were combined and evaporated to give a product which was suspended in THF (21 ml) and a solution of sodium periodate (0.535 g, 2.5 mmol) in water (7 ml) added. The mixture was stirred at room temperature for 1 h. Water and EtOAc were added, then the organic phase was washed with brine, dried (Na₂SO₄) and evaporated to give the title compound (0.522 g, 95%). ¹H NMR (CDCl₃) δ 1.32 (3H, t), 2.95-2.99 (2H, m), 3.05-3.10 (2H, m), 4.32 (2H, q), 5.00 (2H, s), 6.33 (1H, s), 6.96-7.13 (3H, m), 7.81 (1H, s), 7.86 (1H, d), 8.61 (1H, d), 10.15 (1H, s); MS (APCI+) found (M+1)=400; C₂₂H₁₉F₂NO₄ requires 399.

The following intermediates were prepared by the method of intermediate A120.

| No. | Precursor | Structure | Name |
|---|---|---|---|
| A121 | A111 | 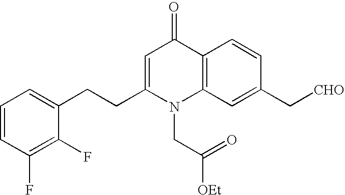 | Ethyl(7-formylmethyl-2-(2-(2,3-di- fluorophenyl)ethyl)-4-oxo-4H-quino- lin-1-yl)acetate |
| A122 | A112 | 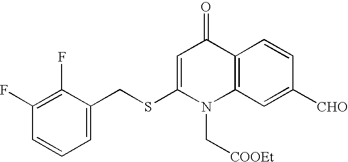 | Ethyl(2-(2,3-difluorobenzylthio)-7-for- myl-4-oxo-4H-quinolin-1-yl)-ace- tate |

### Intermediate A130

Ethyl (7-dimethylaminomethyl-2-(2-(2,3-difluorophenyl)ethyl)-4-oxo-4H-quinolin-1-yl)acetate.

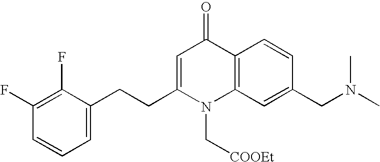

A mixture of intermediate A120 (0.07 g, 0.175 mmol), dimethylamine hydrochloride (0.041 g, 0.525 mmol), sodium triacetoxyborohydride (0.074 g, 0.35 mmol) and acetic acid (0.01 ml) in DMF (1 ml) and THF (3 ml) was stirred at room temperature for 16 h then evaporated under reduced pressure. The residue was stirred with EtOAc and aqueous potassium carbonate. The organic phase was washed with water, then brine, dried (K₂CO₃) and evaporated. Chromatography (silica, CH₂Cl₂/MeOH/NH₃) gave the title compound (0.0644 g, 86%). ¹H NMR (CDCl₃) δ 1.29 (3H, t), 2.25 (6H, s), 2.91-2.96 (2H, m), 3.01-3.07 (2H, m), 3.54 (2H, s), 4.28 (2H, q), 4.94 (2H, s), 6.25 (1H, s), 6.95-7.11 (3H, m), 7.28 (1H, s), 7.32 (1H, d), 8.38 (1H, d); MS (APCI+) found (M+1)=429; C₂₄H₂₆F₂N₂O₃ requires 428.

### Intermediate A131

Ethyl (7-(diethylaminomethyl)-2-(2-(2,3-difluorophenyl)ethyl)-4-oxo-4H-quinolin-1-yl)acetate.

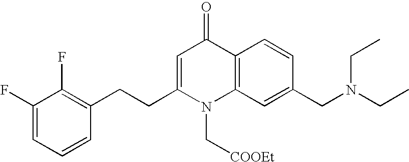

To a mixture of intermediate A120 (0.2 g, 0.5 mmol), acetic acid (0.03 ml) and diethylamine (0.155 ml, 1.5 mmol) in CH₂Cl₂ (15 ml) was added sodium triacetoxyborohydride (0.212 g, 1.0 mmol) portionwise over 10 minutes. After stirring at room temperature for 16 h, saturated sodium bicarbonate was added. The organic phase was washed with water, dried (K₂CO₃) and evaporated. Chromatography (silica, CH₂Cl₂/MeOH/NH₃) gave the title compound (0.2 g, 87%). ¹H NMR (CDCl₃) δ 1.04 (6H, t), 1.29 (3H, t), 2.53 (4H, q), 2.91-2.96 (2H, m), 3.03-3.08 (2H, m), 3.68 (2H, s), 4.28 (2H, q), 4.93 (2H, s), 6.25 (1H, s), 6.96-7.11 (3H, m), 7.32 (1H, d), 7.37 (1H, s), 8.36 (1H, d); MS (APCI+) found (M+1)=457; C₂₆H₃₀F₂N₂O₃ requires 456.

The following intermediates were prepared by the method of intermediate A131.

| No. | Precursor | Structure | Name |
|---|---|---|---|
| A132 | A120 | 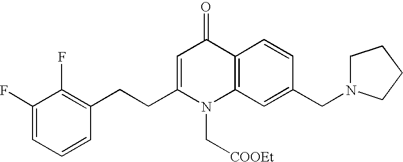 | Ethyl(7-(pyrrolidin-1-ylmethyl)-2-(2-(2,3-di- fluorophenyl)ethyl)-4-oxo-4H-quino- lin-1-yl)acetate |
| A133 | A120 | 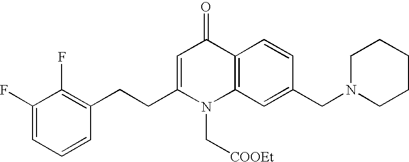 | Ethyl(7-(piperidin-1-ylmethyl)-2-(2-(2,3-di- fluorophenyl)ethyl)-4-oxo-4H-quino- lin-1-yl)acetate |
| A134 | A121 | 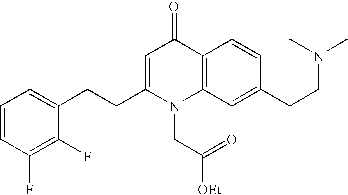 | Ethyl(7-(2-dimethylaminoethyl)-2-(2-(2,3-di- fluorophenyl)ethyl)-4-oxo-4H-quino- lin-1-yl)acetate |
| A135 | A121 | 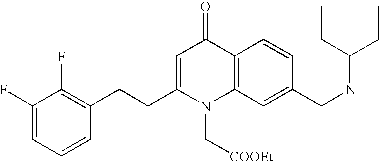 | Ethyl(7-(2-diethylaminoethyl)-2-(2-(2,3-di- fluorophenyl)ethyl)-4-oxo-4H-quino- lin-1-yl)acetate |
| A136 | A121 | 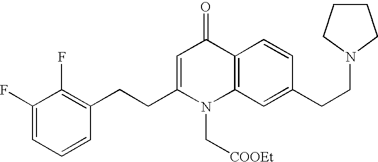 | Ethyl(7-(2-(pyrrolidin-1-yl)ethyl)-2-(2-(2,3-di- fluorophenyl)ethyl)-4-oxo-4H-quino- lin-1-yl)acetate |
| A137 | A122 | 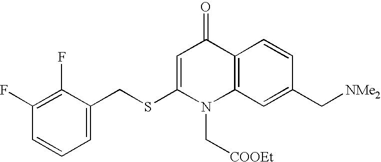 | Ethyl(2-(2,3-difluorobenzylthio)-7-di- methylaminomethyl-4-oxo-4H-quino- lin-1-yl)acetate. |

### Intermediate A140

Ethyl (2-(2,3-difluorobenzylthio)-7-(3-morpholinopropyl)-4-oxo-4H-quinolin-1-yl)acetate

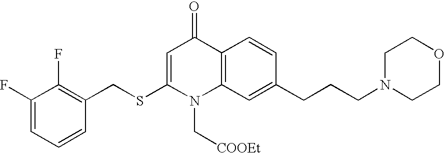

a) To a solution of intermediate A13 (0.4 g, 0.78 mmol) in DMF (20 ml) was added sodium bicarbonate (0.125 g, 1.5 mmol), benzyltriethylammonium chloride (0.18 g, 0.79 mmol), palladium acetate (0.04 g, 0.15 mmol) and allyl alcohol (0.1 ml, 1.5 mmol). The mixture was stirred at 50° C. for 2 h. Further quantities of allyl alcohol (0.1 ml) and palladium acetate (0.04 g) were added, and the mixture was stirred for 2 h at 50° C., then 16 h at room temperature. EtOAc was added and the mixture washed with water, then brine, dried (Na₂SO₄) and evaporated to an oil (0.3 g). This was dissolved in dichloromethane (25 ml) and morpholine (0.2 ml, 2.3 mmol) and acetic acid (0.03 ml) added. To this solution was added sodium triacetoxyborohydride (0.2 g, 0.95 mmol) portionwise over 4 h. After stirring at room temperature for 16 h, the reaction mixture was washed successively with saturated sodium bicarbonate, water, brine, then dried (K₂CO₃) and evaporated. Chromatography (silica, EtOAc/MeOH/NH₃) gave the title compound (0.14 g, 40%). ¹H NMR (CDCl₃) δ 1.28 (3H, t), 1.85 (2H, quintet), 2.35 (2H, t), 2.42 (4H, br t), 2.77 (2H, t), 3.72 (4H, t), 4.27 (2H, q), 4.28 (2H, s), 5.12 (2H, br s), 6.42 (1H, s), 6.98 (1H, s), 7.01-7.16 (3H, m), 7.23 (1H, d), 8.31 (1H, d); MS (APCI+) found (M+1)=517; C₂₇H₃₀F₂N₂O₄S requires 516.

### Intermediate A15

Methyl 4-Hydroxy-2-nitrobenzoate

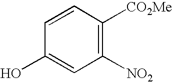

To a suspension of 4-amino-2-nitrobenzoic acid in 20% sulphuric acid (200 ml) at 5-6° C. in an ice-bath was added a solution of sodium nitrite (0.5 g) in water (1.5 ml) dropwise ensuring that the temperature remained ≦6° C. On completion of the addition, a brown homogeneous solution was obtained which was held at 5° C. for 20 min. This solution was added dropwise to 40% sulphuric acid at 130° C. and held at that temperature for 30 min after the addition was complete. The red solution was cooled, extracted with ethyl acetate and the combined organic layers were washed with water and brine and dried over MgSO₄. The solvent was removed under reduced pressure to give a red brown solid (0.58 g). A portion of this solid (0.10 g) was mixed with methanol (5 ml) containing conc. sulphuric acid (1 drop) and heated to reflux for 2 days. The solution was concentrated and partitioned between ethyl acetate and water. The organic layer was washed with further water and brine and dried over MgSO₄. The solvent was removed under reduced pressure and the residue chromatographed on silica gel eluting with ethyl acetate. This gave the title compound as a yellow solid (0.063 g). ¹H NMR (CDCl₃) δ 3.78 (3H, m), 7.11 (1H, dd), 7.24 (1H, d), 7.78 (1H, d), 11.2 (1H, s); MS (APCI−) found (M−1)=196 C₈H₇NO₅ requires 197.

### Intermediate A16

Methyl 4-benzyloxy-2-nitrobenzoate

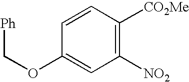

To a mixture of intermediate A15 (7.0 g), triphenylphosphine (11.2 g) and benzyl alcohol (3.68 ml) in dry THF under argon in an ice bath was added diethylazodicarboxylate (7.42 g). The dark red-brown solution so formed was stirred at room temperature overnight and the solvent removed under reduced pressure. The residue was taken up in ethyl acetate, washed with water and brine, dried over MgSO₄ and decolourising charcoal and evaporated under reduced pressure to a brown oil. This material was chromatographed on silica gel to give the title compound (6.17 g). ¹H NMR (CDCl₃) δ 3.87 (3H, m), 5.15 (2H, s), 7.16 (1H, dd), 7.33 (1H, d), 7.3-7.5 (5H, m), 7.77 (1H, d).

### Intermediate A17

Methyl 2-amino-4-benzyloxybenzoate

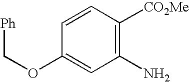

To a mixture of intermediate A16 (6.16 g) and iron powder (17.96 g) in 10% aqueous ethanol (200 ml) was added concentrated hydrochloric acid (1 ml) and the mixture refluxed for 2 h and cooled to room temperature. The mixture was filtered through kieselguhr and the filtrate evaporated under reduced pressure to a black solid. This material was chromatographed on silica using dichloromethane as eluent to give the title compound as a pale yellow solid (2.81 g). ¹H NMR (CDCl₃) δ 3.83 (3H, m), 5.05 (2H, s), 5.76 (2H, br s), 6.18 (1H, d), 6.31 (1H, dd), 7.25-7.5 (5H, m), 7.79 (1H, d).

### Intermediate A18

2-Amino-4-benzyloxybenzoic acid

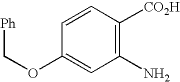

To a solution of intermediate A17 (2.8 g) in methanol (30 ml) was added sodium hydroxide (1.3 g) in water (30 ml) and the mixture was heated to reflux for 5 h. The mixture was concentrated and diluted with water then acidified with 5M hydrochloric acid. The mixture was stirred for 15 min, the solid filtered off and dried to give the title compound (2.4 g). ¹H NMR (d₆-DMSO) δ 5.06 (2H, s), 6.18 (1H, dd), 6.32 (1H, d), 7.2-7.5 (5H, m), 7.62 (1H, d).

The following intermediates were prepared by the method of intermediate A5.

| No. | Precursor | Structure | Name |
|---|---|---|---|
| A21 | 4-Nitroanthranilic acid | 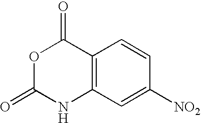 | 7-Nitro-1H-benzo[d][1,3]oxazine-2,4-dione |
| A22 | Int. 18 | 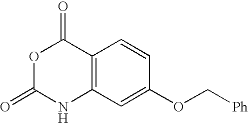 | 7-Benzyloxy-1H-benzo[d][1,3]oxa- zine-2,4-dione |

The following intermediates were prepared by the method of intermediate A6.

| No. | Precursor | Structure | Name |
|---|---|---|---|
| A31 | Int. A21 | 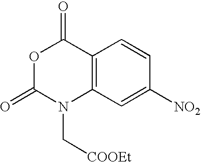 | Ethyl(7-nitro-2,4-dioxo-4H-ben- zo[d][1,3]oxazin-1-yl)acetate |
| A32 | Int. A22 | 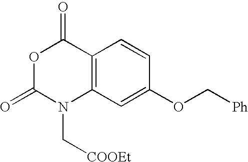 | Ethyl(7-benzyloxy-2,4-dioxo-4H-ben- zo[d][1,3]oxazin-1-yl)acetate |

The following intermediates were prepared by the method of intermediate A8.

| No. | Precursor | Structure | Name |
|---|---|---|---|
| A41 | Int. A31 | 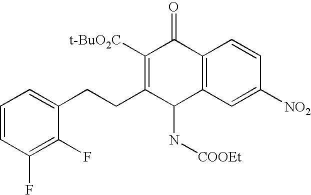 | tert-butyl(7-Nitro-2-(-2-(2,3-di- fluorophenyl)ethyl]-1-ethoxy-carbo- nyl-4-oxo4-Hquinolin-1-yl)-3-car- boxylate. |
| A42 | Int. A32 | 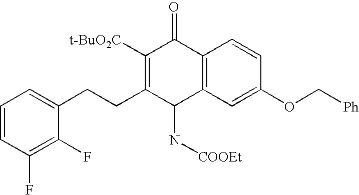 | tert-butyl(7-Benzyloxy-2-(-2-(2,3-di- fluorophenyl)ethyl]-1-ethoxy-car- bonyl-4-oxo4-Hquinolin-1-yl)-3-car- boxylate. |

The following intermediate was prepared by the method of intermediate A9.

| No. | Precursor | Structure | Name |
|---|---|---|---|
| A51 | Int. A41 | 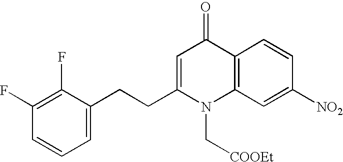 | Ethyl(7-nitro-2-(2-(2,3-difluoro-phe- nyl)ethyl)-4-oxo-4H-quinolin-1-yl)ace- tate |
| A52 | Int. A42 | 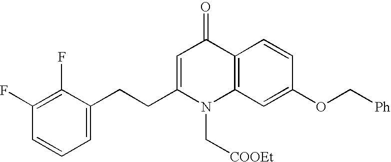 | Ethyl(7-benzyloxy-2-(2-(2,3-di- fluorophenyl)ethyl)-4-oxo-4H-quino- lin-1-yl)acetate |

### Intermediate A61

Ethyl (2-(2-(2,3-difluorophenyl)ethyl)-7-hydroxy-4-oxo-4H-quinolin-1-yl)acetate

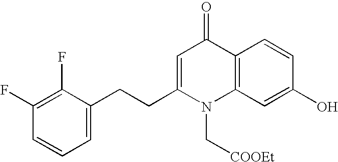

To a solution of intermediate A52 (0.235 g) in dimethylformamide (DMF) (5 ml) was added 10% Pd/C containing 55% water (0.23 g) and the mixture hydrogenated at room temperature and pressure for 0.5 h. The mixture was filtered through kieselguhr and the kieselguhr washed with further DMF. The filtrate was evaporated under reduced pressure to give the title compound as a pale yellow solid (0.192 g). ¹H NMR (d₆-DMSO) δ 1.22 (3H, t), 2.98 (4H, br s), 4.21 (2H, q), 5.09 (2H, br s), 5.89 (1H, d), 6.70 (1H, d), 6.83 (1H, m), 7.05-7.4 (3H, m), 7.98 (1H, d), 10.35 (1H, s).

### Intermediate A71

Ethyl (2-(2-(2,3-difluorophenyl)ethyl)-7-(3-(dimethylamino)propoxy)-4-oxo-4H-quinolin-1-yl) acetate

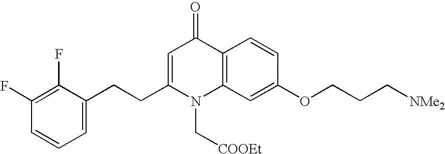

To a solution of intermediate A61 (0.25 g) in dry dimethylformamide (4 ml) at room temperature under argon was added triphenylphosphine (0.508 g) and 3-dimethylaminopropan-1-ol (0.1 g) in dry DMF (2 ml). Diethylazodicarboxylate (0.337 g) in dry DMF (2 ml) was added over 15 min. The dark orange/red mixture was held at room temperature for 21 h and evaporated under reduced pressure. The residue was partitioned between dichloromethane and water. The aqueous layer was extracted with further dichloromethane and the combined organic extracts washed with water and brine and dried over MgSO₄. Evaporation under reduced pressure and chromatography on silica gel using dichloromethane:methanol as eluent gave a gum that was triturated with diethyl ether and hexane (1:1) to give the title compound as an orange solid (0.179 g). ¹H NMR (CDCl₃) δ 1.29 (3H, t), 2.00 (2H, dt), 2.28 (6H, m), 2.48 (2H, t), 2.85-3.15 (4H, m), 4.11 (2H, t), 4.29 (2H, q), 4.85 (2H, s), 6.21 (1H, s), 6.64 (1H, d), 6.9-7.2 (4H, m) 8.35 (1H, d); MS (APCI+) found (M+1)=473; C₂₆H₃₀F₂N₂O₄ requires 472.

The following intermediates were prepared by the method of intermediate A71 from intermediate A61.

| No. | Structure | Name |
|---|---|---|
| A72 | 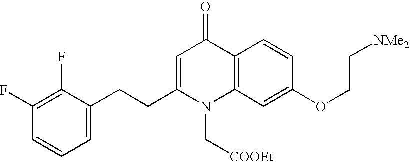 | Ethyl(2-(2-(2,3-difluorophenyl)ethyl)-7-(2-di- methylaminoethoxy)-4-oxo-4H-quinolin-1-yl)ace- tate |
| A73 | 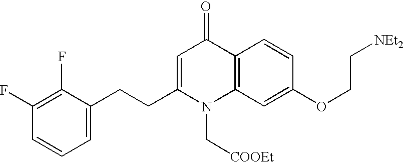 | Ethyl(7-(2-diethylaminoethoxy)-2-(2-(2,3-di- fluorophenyl)ethyl)-4-oxo-4H-quinolin-1-yl)ace- tate |
| A74 | 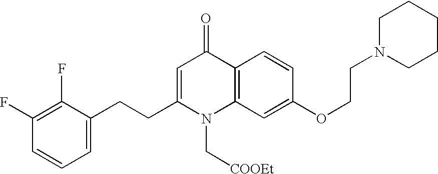 | Ethyl(2-(2-(2,3-difluorophenyl)ethyl)-7-(2-(pipe- ridin-1-yl)ethoxy)-4-oxo-4H-quinolin-1-yl)ace- tate |
| A75 | 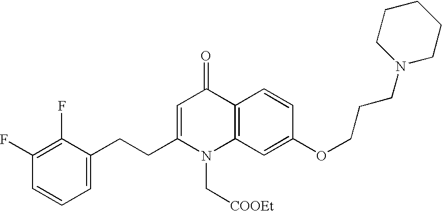 | Ethyl(2-(2-(2,3-difluorophenyl)ethyl)-7-(3-(pipe- ridin-1-yl)propoxy)-4-oxo-4H-quino- lin-1-yl)acetate |
| A76 | 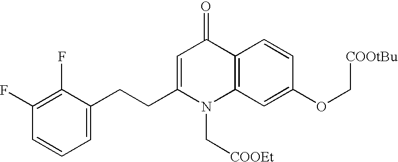 | tert.Butyl2-(2-(2-(2,3-difluorophenyl)-eth- yl))-1-(ethoxycarbonylmethyl)-4-oxo-4H-quino- lin-7-yloxy)acetate |

### Intermediate A85

Ethyl (2-(2-(2,3-difluorophenyl)ethyl)-7-(pyridin-2-ylmethoxy)-4-oxo-4H-quinolin-1-yl) acetate

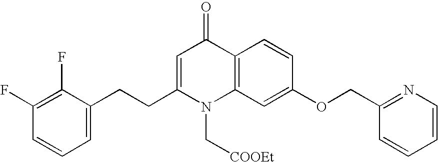

To a suspension of intermediate A61 (0.2 g) in dry DMF (4 ml) under argon was added sodium hydride (0.025 g) at room temperature to form a brown solution. The mixture was stirred at room temperature for 15 min and 2-(bromomethyl)pyridine hydrobromide (0.157 g) added. After stirring at room temperature for 1.5 h, the mixture was evaporated under reduced pressure and partitioned between dichloromethane and dilute brine. The aqueous layer was extracted with further dichloromethane and the combined organic extracts washed with water and brine and dried over MgSO₄. Evaporation under reduced pressure followed by trituration (1:1 diethyl ether:hexane) of the residue so formed gave the title compound as a yellow solid (0.186 g). ¹H NMR (CDCl₃) δ 1.29 (3H, t), 2.8-3.0 (2H, m), 3.0-3.1 (2H, m), 4.26 (2H, q), 4.82 (2H, br s), 5.29 (2H, s), 6.21 (1H, s), 6.78 (1H, d), 6.9-7.15 (4H, m), 7.27 (1H, m), 7.51 (1H, d), 7.74 (1H, dt), 8.37 (1H, d), 8.63 (1H, m); MS (APCI+) found (M+1)=749; C₂₇H₂₄F₂N₂O₄ requires 748.

The following intermediates were prepared by the method of intermediate A85.

| No. | Precursor | Structure | Name |
|---|---|---|---|
| A86 | Int. A61 | 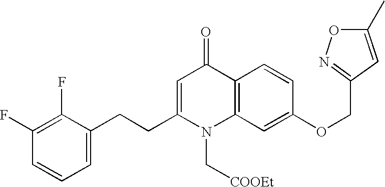 | Ethyl(2-(2-(2,3-difluorophenyl)-eth- yl)-7-(5-methylisoxazol-3-yl-meth- oxy)-4-oxo-4H-quinolin-1-yl)ace- tate |
| A87 | Int. A61 4-bromomethylpipe- ridine.HBr | 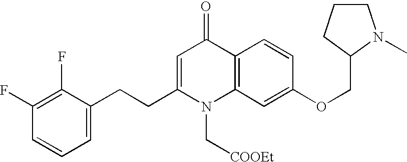 | Ethyl(2-(2-(2,3-difluorophenyl)-eth- yl)-7-(1-methylpyrrolidin-2-yl-meth- oxy)-4-oxo-4H-quinolin-1-yl)ace- tate |

### Intermediate B1

Sodium (2-(2-(2,3-difluorophenyl)ethyl)-7-(3-dimethylamino-propyl)-4-oxo-4H-quinolin-1-yl)acetate.

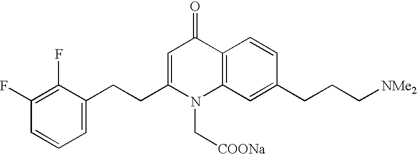

A solution of intermediate A11 (0.29 g, 0.64 mmol) and sodium hydroxide (0.03 g, 0.7 mmol) in dioxan (8 ml) and water (3 ml) was stirred at room temperature for 3 h. The solvent was evaporated to give the title compound (0.3 g, 100%). ¹H NMR (d₆-DMSO) δ 1.74 (2H, m), 2.13 (6H, s), 2.20 (2H, t), 2.69 (2H, t), 2.96 (2H, m), 3.05 (2H, m), 4.50 (2H, s), 5.87 (1H, s), 7.10-7.35 (5H, m), 8.03 (1H, d).

### Intermediate B2

(2-(2-(2,3-difluorophenyl)ethyl)-7-(3-(dimethylamino)propoxy)-4-oxo-4H-quinolin-1-yl)acetic acid hydrochloride

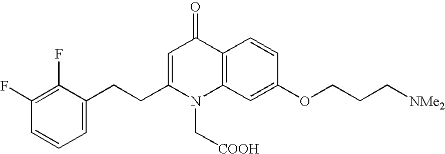

To a stirred solution of intermediate A71 (0.161 g) in methanol (5 ml) was added 0.5M sodium hydroxide (1.37 ml). After 3 h, the mixture was evaporated under reduced pressure, water added and the solution acidified (2M hydrochloric acid) to pH 1 and the precipitate so formed centrifuged to give the title compound (0.137 g). ¹H NMR (d₆-DMSO) δ 2.1-2.3 (2H, br), 2.79 (6H, s), 3.03 (4H, br s), 3.15-3.3 (2H, br), 4.1-4.3 (2H, br), 5.20 (2H, br s), 6.08 (1H, br s), 6.9-7.4 (5H, m), 8.11 (1H, d); MS (APCI−) found (M−1)=443; C₂₄H₂₆F₂N₂O₄ requires 444.

The following intermediate was prepared by the method of intermediate B1.

| No. | Precursor | Structure | Name |
|---|---|---|---|
| B3 | Int. A51 | 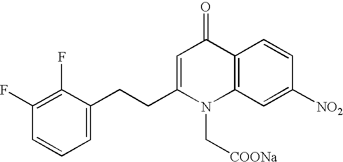 | Sodium(7-nitro-2-(2-(2,3-difluoro-phe- nyl)ethyl)-4-oxo-4H-quinolin-1-yl)ace- tate |
| B20 | Int. A130 | 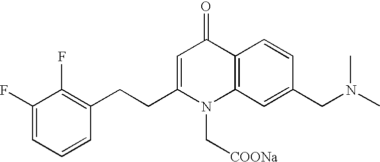 | Sodium(7-(dimethylaminomethyl)-2-(2-(2,3-di- fluorophenyl)ethyl)-4-oxo-4H-quino- lin-1-yl)acetate |
| B21 | Int. A131 | 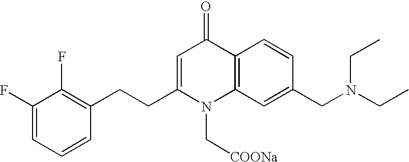 | Sodium(7-(diethylaminomethyl)-2-(2-(2,3-di- fluorophenyl)ethyl)-4-oxo-4H-quino- lin-1-yl)acetate |
| B22 | Int. A132 | 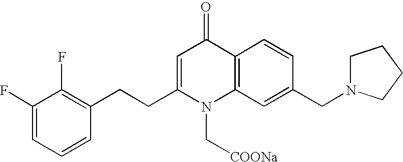 | Sodium(7-((pyrrolidin-1-yl)methyl)-2-(2-(2,3-di- fluorophenyl)ethyl)-4-oxo-4H-quino- lin-1-yl)acetate |
| B23 | Int. A133 | 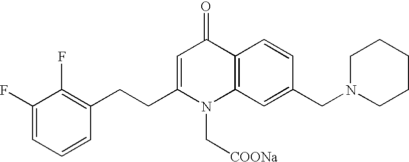 | Sodium(7-((piperidin-1-yl)methyl)-2-(2-(2,3-di- fluorophenyl)ethyl)-4-oxo-4H-quino- lin-1-yl)acetate |
| B24 | Int. A134 | 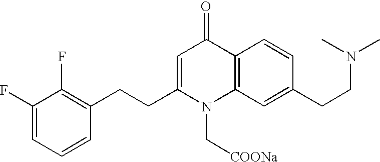 | Sodium(7-(2-dimethylaminoethyl)-2-(2-(2,3-di- fluorophenyl)ethyl)-4-oxo-4H-quino- lin-1-yl)acetate |
| B25 | Int. A135 | 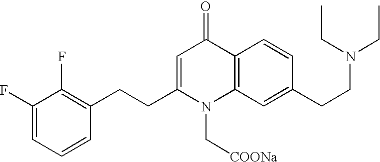 | Sodium(7-(2-diethylaminoethyl)-2-(2-(2,3-di- fluorophenyl)ethyl)-4-oxo-4H-quino- lin-1-yl)acetate |
| B26 | Int. A136 | 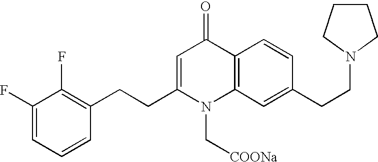 | Sodium(7-(2-(pyrrolidin-1-yl)ethyl)-2-(2-(2,3-di- fluorophenyl)ethyl)-4-oxo-4H-quino- lin-1-yl)acetate |
| B27 | Int. A100 | 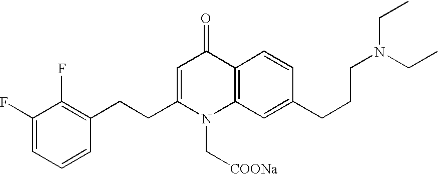 | Sodium(7-(3-diethylaminopropyl)-2-(2-(2,3-di- fluorophenyl)ethyl)-4-oxo-4H-quino- lin-1-yl)acetate |
| B28 | Int. A101 | 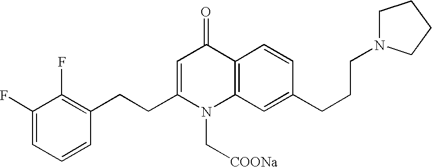 | Sodium(7-(3-(pyrrolidin-1-yl)-pro- pyl)-2-(2-(2,3-difluorophenyl)-eth- yl)-4-oxo-4H-quinolin-1-yl)acetate |
| B29 | Int. A102 | 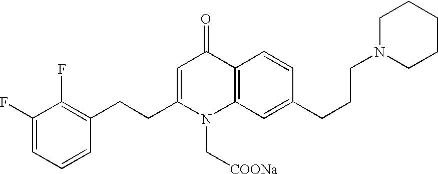 | Sodium(7-(3-(piperidin-1-yl)propyl)-2-(2-(2,3-di- fluorophenyl)ethyl)-4-oxo-4H-quino- lin-1-yl)acetate |
| B30 | Int. A137 | 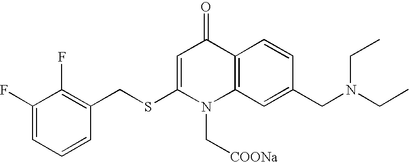 | Sodium(7-diethylaminomethyl)-2-(2,3-di- fluorobenzylthio)-4-oxo-4H-quino- lin-1-yl)acetate |
| B31 | Int. A140 | 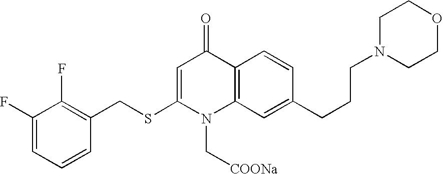 | Sodium(7-(3-(4-morpholino)propyl)-2-(2-(2,3-di- fluorophenyl)ethyl)-4-oxo-4H-quino- lin-1-yl)acetate |

The following intermediates were prepared by the method of intermediate B2.

| No. | Precursor | Structure | Name |
|---|---|---|---|
| B10 | Int. A72 | 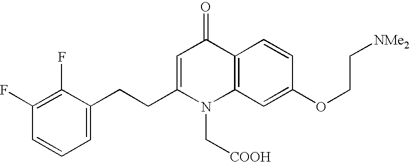 | (2-(2-(2,3-Difluorophenyl)ethyl)-7-(2-di- methylaminoethoxy)-4-oxo-4H-quino- lin-1-yl)acetic acid |
| B11 | Int. A73 | 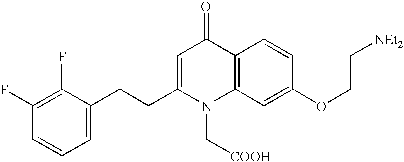 | (7-(2-Diethylaminoethoxy)-2-(2-(2,3-di- fluorophenyl)ethyl)-4-oxo-4H-quino- lin-1-yl)acetic acid |
| B12 | Int. A74 | 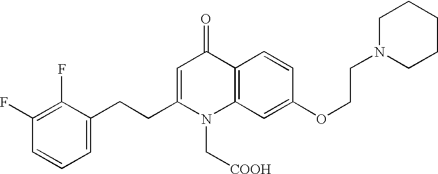 | (2-(2-(2,3-Difluorophenyl)ethyl)-7-(2-(pipe- ridin-1-yl)ethoxy)-4-oxo-4H-quino- lin-1-yl)acetic acid |
| B13 | Int. A75 | 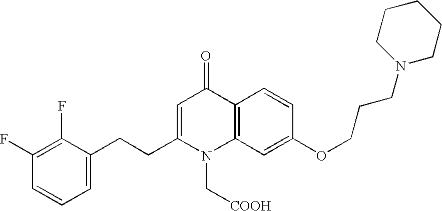 | (2-(2-(2,3-Difluorophenyl)ethyl)-7-(3-(pipe- ridin-1-yl)propoxy)-4-oxo-4H-quino- lin-1-yl)acetic acid |
| B14 | Int. A76 | 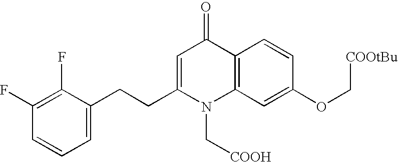 | (7-(tert.Butoxycarbonylmethoxy)-2-(2-(2,3-di- fluorophenyl)ethyl)-4-oxo-4H-quino- lin-1-yl)acetic acid |
| B15 | Int. A85 | 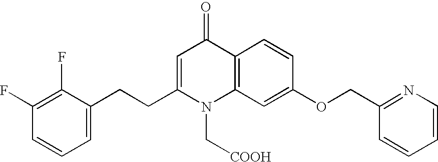 | (2-(2-(2,3-Difluorophenyl)ethyl)-7-(py- ridin-1-ylmethoxy)-4-oxo-4H-quino- lin-1-yl)acetic acid |
| B16 | Int. A86 | 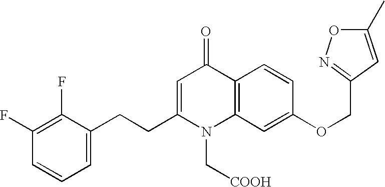 | (2-(2-(2,3-Difluorophenyl)ethyl)-7-(5-methyl- isoxazol-3-ylmethoxy)-4-oxo-4H-quino- lin-1-yl)acetic acid |
| B17 | Int. A87 | 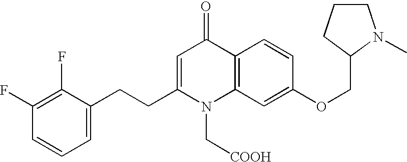 | (2-(2-(2,3-Difluorophenyl)ethyl)-7-(1-meth- ylpyrrolidin-2-ylmethoxy)-4-oxo-4H-quino- lin-1-yl)acetic acid |
| B18 | Int. A52 | 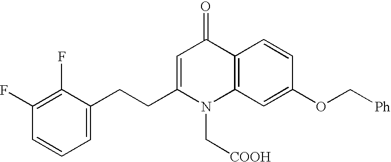 | (7-Benzyloxy-2-(2-(2,3-difluoro-phe- nyl)ethyl)-4-oxo-4H-quinolin-1-yl)ace- tic acid |

The following amines are known in the literature.

| No. | Reference | Structure | Name |
|---|---|---|---|
| C1 | WO00/66567 | 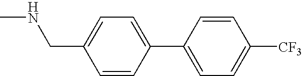 | 4-(4-Trifluoromethylphenyl)-N-meth- ylbenzylamine |
| C2 | WO01/60805 | 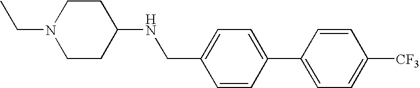 | N-(1-Ethyl-piperidin-2-yl)-4-(4-tri- fluoromethyl-phe- nyl)benzylamine |

### Intermediate C3

4-(4-Trifluoromethylphenyl)-N-ethylbenzylamine

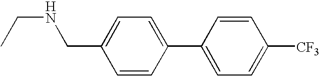

To a solution of 4-(4-trifluoromethylphenyl)benzaldehyde (5.0 g, 20 mmol) in CH₂C₂ (100 ml) was added a solution of ethylamine in THF (2M, 20 ml, 40 mmol). 4Å molecular sieves (15 g) were added and the mixture stirred gently for 16 h. The mixture was filtered through celite and the filtrate evaporated. The residue was dissolved in ethanol (200 ml), and sodium borohydride (1.13 g, 30 mmol) was added portionwise over 10 minutes. The mixture was stirred at room temperature for 1 h, then concentrated. CH₂Cl₂ and water were added, the organic phase was washed with water, dried (K₂CO₃) and evaporated to give the title compound as a white solid. (4.9 g, 88%). ¹H NMR (CDCl₃) δ 1.16 (3H, t), 2.72 (2H, q), 3.85 (2H, s), 7.43 (2H, d), 7.56 (2H, d), 7.68 (4H, s); MS (APCI+) found (M+1)=280; C₁₆H₁₆F₃N requires 279.

The following intermediate was prepared by the method of intermediate C3.

| No. | Structure | Name |
|---|---|---|
| C4 | 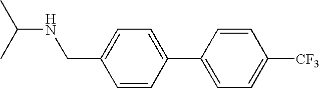 | 4-(4-Trifluoromethylphenyl)-N-iso- propylbenzylamine |

### Example 1

N-Methyl-2-(2-(2-(2,3-difluorophenyl)ethyl)-7-(3-dimethylaminoprop-1-yl)-4-oxo-4H-quinolin-1-yl)-N-(4-(4-trifluoromethylphenyl)benzyl)acetamide bitartrate

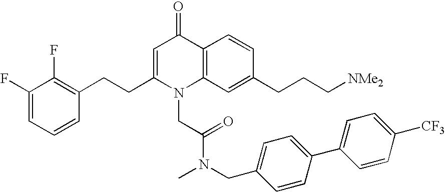

O-(7-Azabenzotriazol-1-yl)-N,N,N′,N′-tetramethyluronium hexafluorophosphate (HATU) (0.187 g, 0.6 mmol) was added to a mixture of intermediate B1 (0.195 g, 0.43 mmol), amine C1 (0.115 g, 0.43 mmol) and diisopropylethylamine (0.18 ml, 1.04 mmol) in dimethylformamide (10 ml) and the resultant solution stirred for 2 h. The solvent was evaporated and the residue diluted with dichloromethane (30 ml) and washed successively with saturated ammonium chloride and saturated sodium bicarbonate. The organic layer was dried (K₂CO₃) and the solvent evaporated. The residue was purified by flash chromatography NH₃/MeOH/CH₂Cl₂). The amine (0.18 g, 0.267 mmol) was dissolved in methanol (10 ml) and tartaric acid (0.04 g, 0.267 mmol) added. After stirring for 15 min the solvent was evaporated and the residue triturated from diethyl ether to afford the title compound (0.215 g). ¹H NMR (d₆-DMSO) δ 1.81 (2H, m), 2.19 (6H, 2x s), 2.27 (2H, m), 2.65 (6H, m), 3.2 (3H, 2x s), 4.12 (2H s), 4.63, 4.81 (2H, 2x s), 5.29, 5.39 (2H, 2x br s), 5.99 (1H, 2x s), 7.03-7.88 (13H, m), 8.07 (1H, 2x d); MS (APCI+) found (M+1)=676; C₃₉H₃₈F₅N₃O₂ requires 675.

The following examples were prepared by the method of example 1 using either the parent acid or its sodium salt.

| Ex. | Precursors | Structure | Name |
|---|---|---|---|
| 2 | Int. B2 Amine C1 | 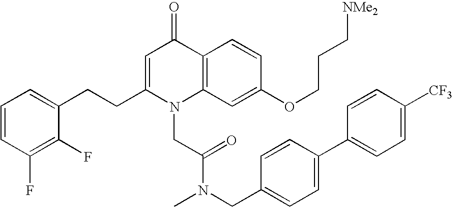 | 2-(2-(2-(2,3-Difluorophenyl)-eth- yl)-4-oxo-7-(3-(dimethyl-a- mino)propoxy)-4H-quinolin-1-yl)-N-meth- yl-N-(4-(4-trifluorometh- ylphenyl)benzyl)acetamide bitartrate |
| 3 | Int. B3 Amine C1 | 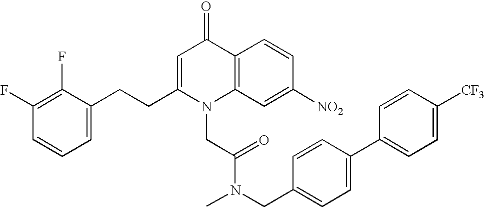 | N-Methyl-2-(2-(2-(2,3-difluorophe- nyl)ethyl)-7-nitro-4-oxo-4H-quino- lin-1-yl)-N-(4-(4-trifluoro-meth- ylphenyl)benzyl) acetamide |
| 4 | Int. B10 Amine C1 | 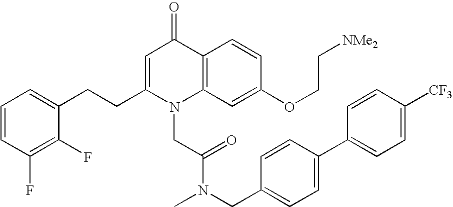 | 2-(2-(2-(2,3-Difluorophenyl)eth- yl)-7-(2-dimethylamino-eth- oxy)-4-oxo-4H-quinolin-1-yl)-N-meth- yl-N-(4-(4-trifluoro-meth- ylphenyl)benzyl)acetamide bitartrate |
| 5 | Int. B11 Amine C1 | 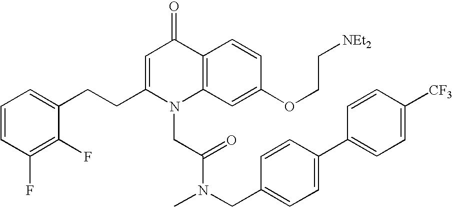 | 2-(7-(2-Diethylaminoethoxy)-2-(2-(2,3-di- fluorophenyl)ethyl)-4-oxo-4H-quino- lin-1-yl)-N-meth- yl-N-(4-(4-trifluoromethylphe- nyl)benzyl)acetamide bitartrate |
| 6 | Int. B12 Amine C1 | 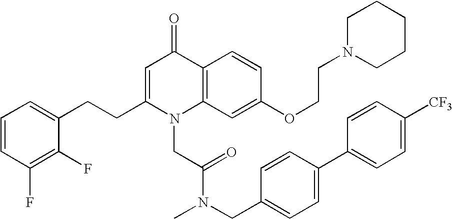 | 2-(2-(2-(2,3-Difluorophenyl)-eth- yl)-4-oxo-7-(2-(piperidin-1-yl)eth- oxy)-4H-quinolin-1-yl)-N-meth- yl-N-(4-(4-trifluoromethylphe- nyl)benzyl)acetamide bitartrate |
| 7 | Int. B13 Amine C1 | 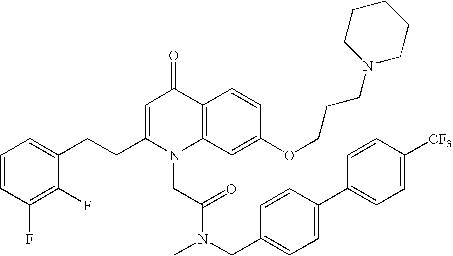 | 2-(2-(2-(2,3-Difluorophenyl)-eth- yl)-4-oxo-7-(3-(piperidin-1-yl)pro- poxy)-4H-quinolin-1-yl)-N-meth- yl-N-(4-(4-trifluorometh- ylphenyl)benzyl)acetamide bitartrate |
| 8 | Int. B14 Amine C1 | 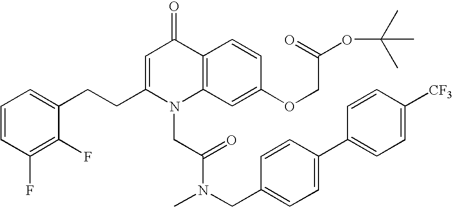 | tert.Butyl2-(2-(2-(2,3-difluoro-phe- nyl)ethyl))-1-(N-(4-(4-tri- fluoromethylphenyl)benzyl)-N-meth- yl-aminocarbonyl-meth- yl)-4-oxo-4H-quinolin-7-yl- oxy)acetate |
| 9 | Int. B15 Amine C1 | 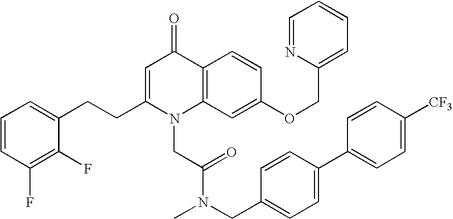 | 2-(2-(2-(2,3-Difluorophenyl)-eth- yl)-4-oxo-7-(pyridin-2-yl-meth- oxy)-4H-quinolin-1-yl)-N-meth- yl-N-(4-(4-trifluoromethylphe- nyl)benzyl)acetamide hydrochloride |
| 10 | Int. B16 Amine C1 | 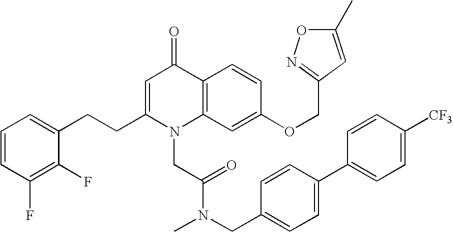 | 2-(2-(2-(2,3-Difluorophenyl)-eth- yl)-7-(5-methylisoxazol-3-yl- methoxy)-4-oxo-4H-quinolin-1-yl)-N-meth- yl-N-(4-(4-tri- fluoromethylphenyl)ben- zyl)acetamide |
| 11 | Int. B17 Amine C1 | 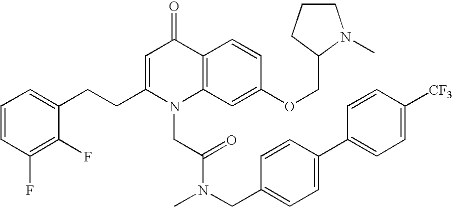 | 2-(2-(2-(2,3-Difluorophenyl)-eth- yl)-7-(1-methylpyrrolidin-2-yl- methoxy)-4-oxo-4H-quinolin-1-yl)-N-meth- yl-N-(4-(4-tri- fluoromethylphenyl)ben- zyl)acetamide bitartrate |
| 12 | Int. B18 Amine C1 | 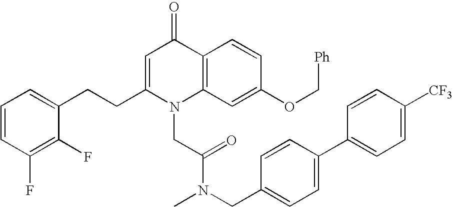 | 2-(7-Benzyloxy-2-(2-(2,3-di- fluorophenyl)ethyl)-4-oxo-4-quino- lin-1-yl)-N-methyl-N-(4-(4-tri- fluoromethylphenyl)ben- zyl)acetamide |
| 13 | Int. B18 Amine C2 | 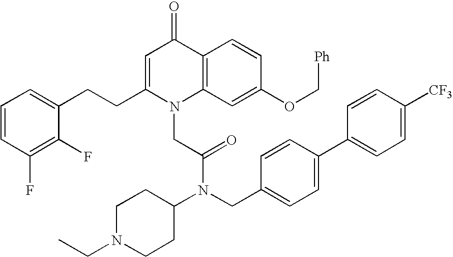 | 2-(7-Benzyloxy-2-(2-(2,3-di- fluorophenyl)ethyl)-4-oxo-4-quino- lin-1-yl)-N-(1-ethyl-pipe- ridin-4-yl)-N-(4-(4-tri- fluoromethylphenyl)benzyl)-ace- tamide bitartrate |
| 20 | Int. B20 Amine C1 | 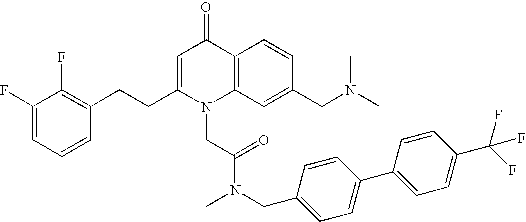 | 2-(7-(Dimethylaminomethyl)-2-(2-(2,3-di- fluorophenyl)ethyl)-4-oxo-4H-quino- lin-1-yl)-N-meth- yl-N-(4-(4-trifluoromethylphe- nyl)benzyl)acetamide bitartrate |
| 21 | Int. B21 Amine C1 | 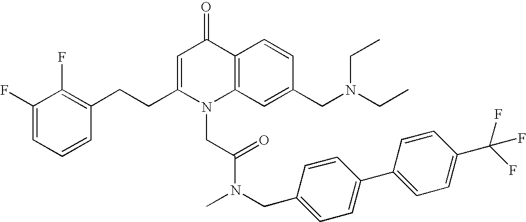 | 2-(7-(Diethylaminomethyl)-2-(2-(2,3-di- fluorophenyl)ethyl)-4-oxo-4H-quino- lin-1-yl)-N-meth- yl-N-(4-(4-trifluoromethylphe- nyl)benzyl)acetamide bitartrate |
| 22 | Int. B21 Amine C3 | 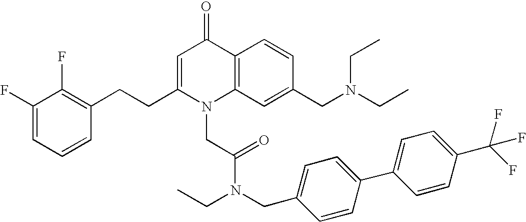 | 2-(7-(Diethylaminomethyl)-2-(2-(2,3-di- fluorophenyl)ethyl)-4-oxo-4H-quino- lin-1-yl)-N-ethyl-N-(4-(4-tri- fluoromethylphe- nyl)benzyl)acetamide bitartrate |
| 23 | Int. B21 Amine C4 | 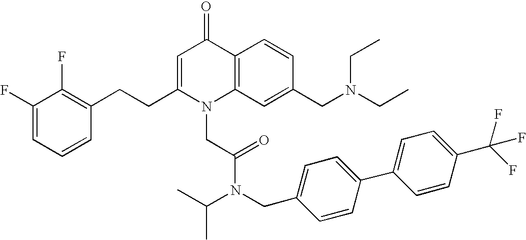 | 2-(7-(Diethylaminomethyl)-2-(2-(2,3-di- fluorophenyl)ethyl)-4-oxo-4H-quino- lin-1-yl)-N-iso- propyl-N-(4-(4-trifluorometh- ylphenyl)benzyl)acetamide bitartrate |
| 24 | Int. B22 Amine C3 | 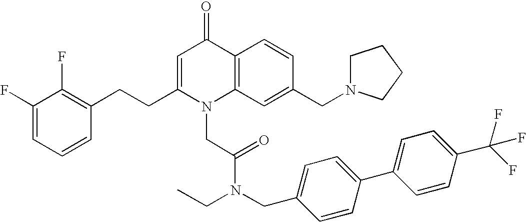 | 2-(7-((Pyrrolidin-1-yl)methyl)-2-(2-(2,3-di- fluorophenyl)ethyl)-4-oxo-4H-quino- lin-1-yl)-N-eth- yl-N-(4-(4-trifluoromethyl- phenyl)benzyl)acetamide bitartrate |
| 25 | Int. B23 Amine C3 | 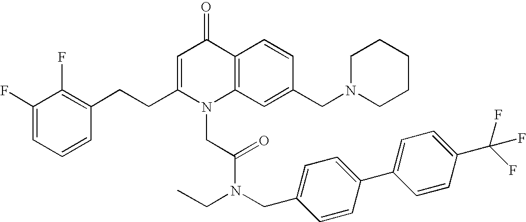 | 2-(7-((Piperidin-1-yl)methyl)-2-(2-(2,3-di- fluorophenyl)ethyl)-4-oxo-4H-quino- lin-1-yl)-N-ethyl-N-(4-(4-tri- fluoromethylphenyl)ben- zyl)acetamide bitartrate |
| 26 | Int. B24 Amine C1 | 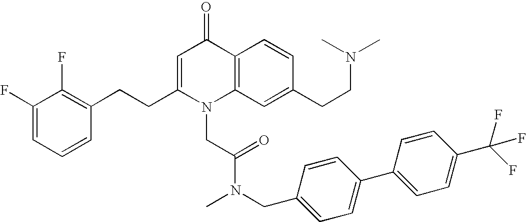 | 2-(7-(2-Dimethylaminoethyl)-2-(2-(2,3-di- fluorophenyl)ethyl)-4-oxo-4H-quino- lin-1-yl)-N-meth- yl-N-(4-(4-trifluoromethyl- phenyl)benzyl)acetamide bitartrate |
| 27 | Int. B25 Amine C1 | 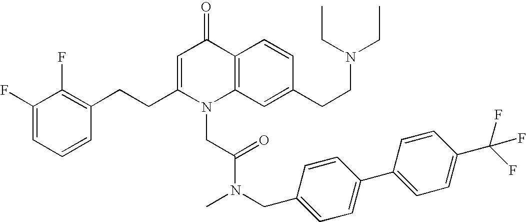 | 2-(7-(2-Diethylaminoethyl)-2-(2-(2,3-di- fluorophenyl)ethyl)-4-oxo-4H-quino- lin-1-yl)-N-meth- yl-N-(4-(4-trifluoromethyl- phenyl)benzyl)acetamide bitartrate |
| 28 | Int. B26 Amine C1 | 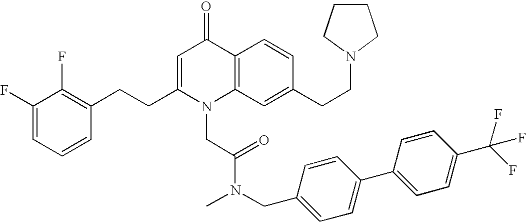 | 2-(7-(2-(Pyrrolidin-1-yl)ethyl)-2-(2-(2,3-di- fluorophenyl)ethyl)-4-oxo-4H-quino- lin-1-yl)-N-meth- yl-N-(4-(4-trifluoromethylphe- nyl)benzyl)acetamide bitartrate |
| 29 | Int. B27 Amine C1 | 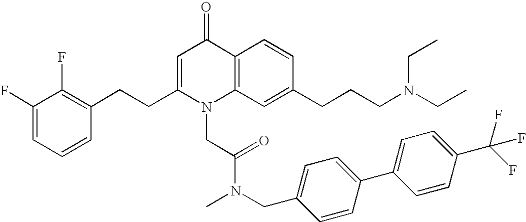 | 2-(7-(3-Diethylaminopropyl)-2-(2-(2,3-di- fluorophenyl)ethyl)-4-oxo-4H-quino- lin-1-yl)-N-meth- yl-N-(4-(4-trifluoromethylphe- nyl)benzyl)acetamide bitartrate |
| 30 | Int. B28 Amine C1 | 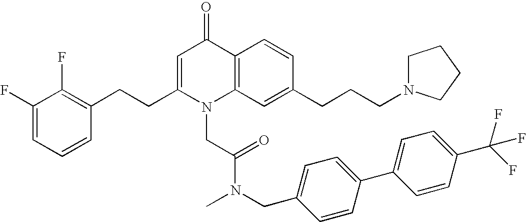 | 2-(7-(3-(Pyrrolidin-1-yl)pro- pyl)-2-(2-(2,3-di- fluorophenyl)ethyl)-4-oxo-4H-quino- lin-1-yl)-N-methyl-N-(4-(4-tri- fluoromethylphe- nyl)benzyl)acetamide bitartrate |
| 31 | Int. B29 Amine C3 | 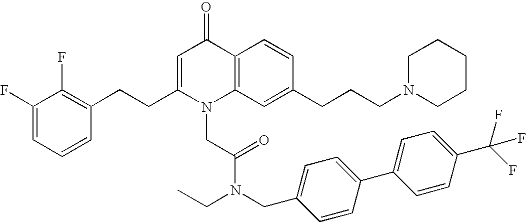 | 2-(7-(3-(Piperidin-1-yl)propyl)-2-(2-(2,3-di- fluorophenyl)ethyl)-4-oxo-4H-quino- lin-1-yl)-N-eth- yl-N-(4-(4-trifluoromethylphe- nyl)benzyl)acetamide bitartrate |
| 32 | Int. B29 Amine C4 | 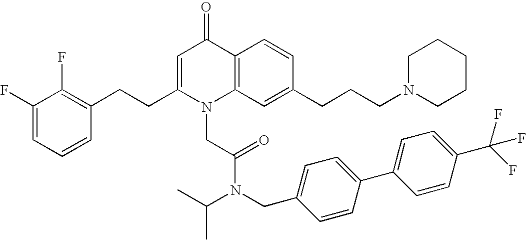 | 2-(7-(3-(Piperidin-1-yl)propyl)-2-(2-(2,3-di- fluorophenyl)ethyl)-4-oxo-4H-quino- lin-1-yl)-N-iso- propyl-N-(4-(4-trifluorometh- ylphenyl)benzyl)acetamide bitartrate |
| 33 | Int. B30 Amine C1 | 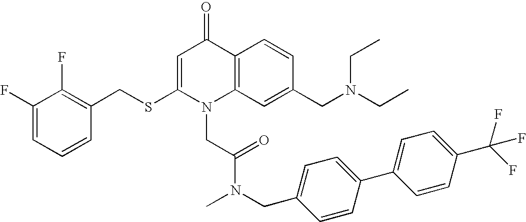 | 2-(7-(Diethylaminomethyl)-2-(2,3-di- fluorobenzylthio)-4-oxo-4H-quino- lin-1-yl)-N-methyl-N-(4-(4-tri- fluoromethylphenyl)-ben- zyl)acetamide bitartrate |
| 34 | Int. B31 Amine C1 | 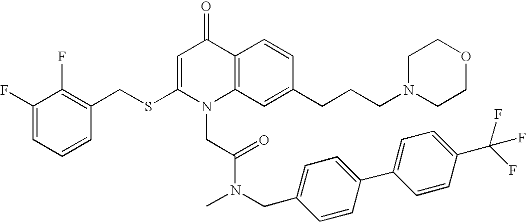 | 2-(7-(3-(4-Morpholino)propyl)-2-(2,3-di- fluorobenzylthio)-4-oxo-4H-quino- lin-1-yl)-N-meth- yl-N-(4-(4-trifluoromethyl- phenyl)benzyl)acetamide hydrochloride |

### Example 15

2-(2-(2-(2,3-Difluorophenyl)ethyl)-7-hydroxy-4-oxo-4H-quinolin-1-yl)-N-methyl-N-(4-(4-trifluoromethylphenyl)benzyl)acetamide

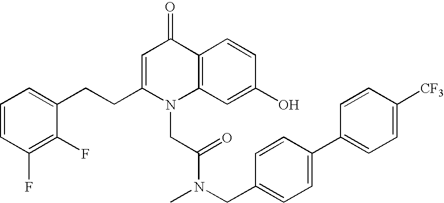

To a solution of example 12 (0.382 g) in dimethylformamide (DMF) (30 ml) was added 10% Pd/C (paste containing 54% water) and the mixture hydrogenated at room temperature for 2.5 h. The mixture was diluted with further DMF (70 ml), warmed to dissolve any precipitated product and filtered through Celite and a small plug of fine silica gel. The solvent was removed under reduced pressure and the residue triturated with diethyl ether and dried to give the title compound (0.242 g). ¹H NMR (d₆-DMSO) δ 2.75-3.1 (4H, m), 3.21+3.31 (3H, 2x s), 4.6+4.87 (2H, 2xbr s), 5.18+5.26 (2H, 2xbr s), 5.89+5.91 (1H, 2xs), 6.55-6.9 (2H, m), 7.05-7.5 (5H, m), 7.55-7.75 (2H, m) 7.75-7.90 (4H, m), 7.9-8.05 (1H, m), 10.23+10.28 (1H, 2xs).

The following example was prepared by the method of Example 15 but using ethanol as solvent.

| Ex. | Precursor | Structure | Name |
|---|---|---|---|
| 16 | Ex. 13 | 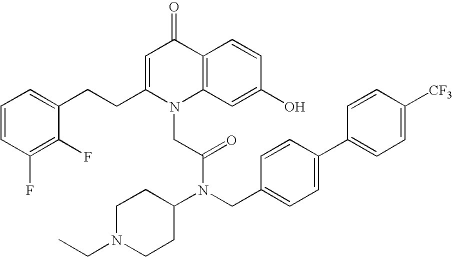 | 2-(2-(2-(2,3-Difluorophenyl)eth- yl)-7-hydroxy-4-oxo-4H-quino- lin-1-yl)-N-(1-ethyl-pipe- ridin-4-yl)-N-(4-(4-trifluoro- methylphenyl)benzyl)acetamide bitartrate |

### Example 17

2-(2-(2-(2,3-Difluorophenyl)ethyl))-1-(N-(4-(4-trifluoromethyl-phenyl)benzyl)-N-methyl-aminocarbonylmethyl)-4-oxo-4H-quinolin-7-yloxy)acetic acid

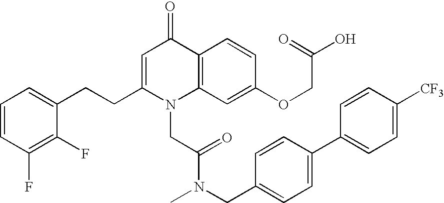

To a solution of example 8 (0.135 g) in dichloromethane (3 ml) was added trifluoroacetic acid (0.5 ml) and the solution stirred for 66 h at room temperature. The solvent was removed under reduced pressure and the residue triturated with diethyl ether to give the title compound (0.115 g). ¹H NMR (d₆-DMSO) δ 2.8-3.1+3.22 (7H, m+s), 4.63+4.75-5.0 (4H, br s+m), 5.2-5.5 (2H, m), 6.01+6.04 (1H 2xs), 6.8-7.6 (7H, 2x s), 7.55-7.95 (6H, m), 8.05-8.2 (1H, m); MS (APCI−) found (M−1)=663 (weak); C₃₆H₂₉F₅N₃O₅ requires 664.

Biological Data

1. Screen for Lp-PLA₂ Inhibition.

Enzyme activity was determined by measuring the rate of turnover of the artificial substrate (A) at 37° C. in 50 mM HEPES (N-2-hydroxyethylpiperazine-N′-2-ethanesulphonic acid) buffer containing 150 mM NaCl, pH 7.4.

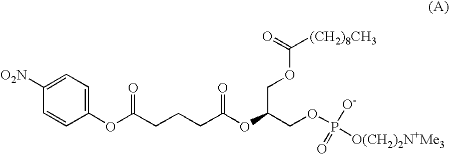

Assays were performed in 96 well titre plates. Recombinant Lp-PLA₂ was purified to homogeneity from baculovirus infected Sf9 cells, using a zinc chelating column, blue sepharose affinity chromatography and an anion exchange column. Following purification and ultrafiltration, the enzyme was stored at 6 mg/ml at 4° C. Assay plates of compound or vehicle plus buffer were set up using automated robotics to a volume of 170 μl. The reaction was initiated by the addition of 20 μl of 10× substrate (A) to give a final substrate concentration of 20 μM and 10 μl of diluted enzyme to an approximate final 0.1 nM Lp-PLA₂. The reaction was followed at 405 nm and 37° C. for 20 minutes using a plate reader with automatic mixing. The rate of reaction was measured as the rate of change of absorbance.

Results

The compounds described in the Examples were tested as described above and had IC₅₀ values in the range <0.1 to 100 nM.

1. A compound of formula (I): 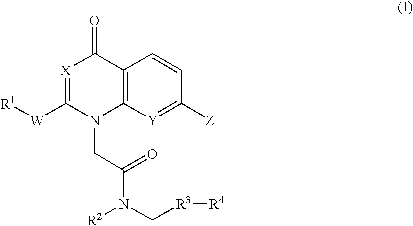
in which:
R¹ is an aryl group, optionally substituted by 1, 2, 3 or 4 substituents which may be the same or different selected from the group consisting of C₍₁₋₆₎alkyl, C₍₁₋₆₎alkoxy, C₍₁₋₆₎alkylthio, hydroxy, halogen, CN, mono to perfluoro-C₍₁₋₄₎alkyl, mono to perfluoro-C₍₁₋₄₎alkoxyaryl, and arylC₍₁₋₄₎alkyl;
R² is hydrogen, C₍₁₋₆₎alkyl which may be unsubstituted or substituted by 1, 2 or 3 substituents selected from the group consisting of hydroxy, halogen, OR⁵, COR⁵, carboxy, COOR⁵, CONR⁷R⁸, NR⁷R⁸, NR⁵COR⁶, mono- or di-(hydroxyC₍₁₋₆₎alkyl)amino and N-hydroxyC₍₁₋₆₎alkyl-N—C₍₁₋₆₎alkylamino; or
R² is Het-C₍₀₋₄₎alkyl in which Het is a 5- to 7-membered heterocyclyl ring comprising N and optionally O or S, and in which N may be substituted by COR⁵, COOR⁵, CONR⁷R⁸, or C₍₁₋₆₎alkyl optionally substituted by 1, 2 or 3 substituents selected from the group consisting of hydroxy, halogen, OR⁵, COR⁵, carboxy, COOR⁵, CONR⁷R⁸ and NR⁷R⁸;
R³ is an aryl or a heteroaryl ring optionally substituted by 1, 2, 3 or 4 substituents which may be the same or different selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁵, carboxy, COOR⁵, NR⁵COR⁶, CONR⁷R⁸, SO₂NR⁷R⁸, NR⁵SO₂R⁶, NR⁷R⁸, mono to perfluoro-C₍₁₋₄₎alkyl and mono to perfluoro-C₍₁₋₄₎alkoxy;
R⁴ is an aryl or a heteroaryl ring which is optionally substituted by 1, 2, 3 or 4 substituents which may be the same or different selected from the group consisting of C₍₁₋₆₎alkyl, C₍₁₋₆₎alkoxy, C₍₁₋₆₎alkylthio, C₍₁₋₆₎alkylsulfonyl, arylC₍₁₋₆₎alkoxy, hydroxy, halogen, CN, COR⁵, carboxy, COOR⁵, CONR⁷R⁸, NR⁵COR⁶, SO₂NR⁷R⁸, NR⁵SO₂R⁶, NR⁷R⁸, mono to perfluoro-C₍₁₋₄₎alkyl and mono to perfluoro-C(₁₋₄₎alkoxy, or C₍₅₋₁₀₎alkyl;
W is a C₍₂₋₄₎alkylene group, optionally substituted by 1, 2 or 3 substituents selected from the group consisting of methyl and ethyl, CH═CH, (CH₂)ₙS or (CH₂)ₙO where n is 1, 2 or 3;
X and Y are independently CH or N;
Z is NO₂, NR⁵R⁹, OR⁹, SR⁹, SOR⁹, SO₂R⁹ or R¹⁰;
R⁵ and R⁶ are independently hydrogen or C₍₁₋₁₂₎alkyl,;
R⁷ and R⁸ which may be the same or different are hydrogen, or C₍₁₋₁₂₎alkyl, or R⁷ and R⁸ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl;
R⁹ is hydrogen or C₍₁₋₆₎alkyl optionally substituted by 1, 2 or 3 substituents which may be the same or different selected from the group consisting of hydroxy, halogen, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or differentselected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from the group consisting of hydroxy, halogen, OR⁵, COR⁵, carboxy, COOR⁵, CONR⁷R⁸ and NR⁷R⁸; and
R¹⁰ is C₍₁₋₆₎alkyl optionally substituted by 1, 2 or 3 substituents which may be the same or different selected from the group consisting of hydroxy, halogen, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different and is selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents and is selected from the group consisting of hydroxy, halogen, OR⁵, COR⁵, carboxy, COOR⁵, CONR⁷R⁸ or NR⁷R⁸; or
R¹⁰ is a 5- to 7-membered heterocyclic ring optionally substituted by COR⁵, COOR⁵, CONR⁷R⁸, or C₍₁₋₆₎alkyl optionally substituted by 1, 2 or 3 substituents selected from the group consisting of hydroxy, halogen, OR⁵, COR⁵, carboxy, COOR⁵, CONR⁷R⁸ or NR⁷R⁸; and pharmaceutically acceptable salts thereof
with the provisos that:
Z is not amino and that the compound of formula (I) is not:
N-(2-diethylaminoethyl)-2-(2-(2-(2,3-difluorophenyl)-ethyl)-7-trifluoromethyl-4-oxo-4H-quinazolin-1-yl)-N-(4′-trifluoromethyl-biphenyl-4-ylmethyl)acetamide;
N-(2-diethylaminoethyl)-2-(2-(2-(2,3-difluorophenyl)-ethyl)-7-methyl-4-oxo-4H-quinazolin-1-yl)-N-(4′-trifluoromethyl-biphenyl-4-ylmethyl)acetamide;
N-(1-ethylpiperidin-4-yl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide;
N-(2-diethylaminoethyl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide;
N-(1-methylpiperidin-4-yl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide;
N-(1-isopropylpiperidin-4-yl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide; or
N-(1-(2-methoxyethyl)piperidin-4-yl)-2-[2-(2-(2,3-difluorophenyl)ethyl)-7-methyl-4-oxo-4H-[1,8]naphthyridin-1-yl]-N-(4′-trifluoromethylbiphenyl-4-ylmethyl)acetamide.

2. A compound according to claim 1 wherein R′ is phenyl optionally substituted by halogen, C₍₁₋₆₎alkyl, trifluoromethyl or C₍₁₋₆₎alkoxy.

3. A compound according to claim 1 wherein R² is hydrogen, methyl, ethyl, isopropyl, 2-(diethylamino)ethyl, 2-(piperidin-1-yl)ethyl, 2-(pyrrolidin-1-yl)ethyl, 1-(2-methoxyethyl)piperidin-4-yl, 1-methylpiperidin-4-yl, 1-ethyl-piperidin-4-yl or 1-ethyl-pyrrolidin-2-ylmethyl.

4. A compound according to claim 1 wherein R³ is phenyl.

5. A compound according to claim 1 wherein R⁴ is phenyl optionally substituted by halogen, trifluoromethyl or ethyl.

6. A compound according to claim 1 wherein W is (CH₂)ₙS or CH₍₂₋₄₎alkylene.

7. A compound according to claim 1 wherein Z is NO₂, OR⁹ or R¹⁰.

8. A compound according to claim 7 wherein Z is hydroxy, nitro, mono or di-N—C₍₁₋₆₎alkylaminoC₍₁₋₆₎alkyl, mono or di-N—C₍₁₋₆₎alkylaminoC₍₁₋₆₎alkoxy, carboxyC₍₁₋₆₎alkoxy or an ester thereof, or arylC₍₁₋₆₎alkoxy, arylC₍₁₋₆₎alkyl, heteroarylC₍₁₋₆₎alkoxy, heteroarylC₍₁₋₆₎alkyl, 5- to 7-membered heterocyclylC₍₁₋₆₎alkoxy optionally substituted by C₍₁₋₆₎alkyl, or 5- to 7-membered heterocyclylC₍₁₋₆₎alkyl optionally substituted by C₍₁₋₆₎alkyl.

9. A compound according to claim 7 wherein when Z includes an aryl, heteroaryl or heterocyclyl ring, said ring is selected from benzyl, pyridinyl, isoxazolyl, piperidinyl, pyrrolidinyl and morpholino.

10. A compound according to claim 1 which is:
N-methyl-2-(2-(2-(2,3-difluorophenyl)ethyl)-7-(3-dimethylaminoprop-1-yl)-4-oxo-4H-quinolin-1-yl)-N-(4-(4-trifluoromethylphenyl)benzyl)acetamide bitartrate;
2-(2-(2-(2,3-difluorophenyl)ethyl)-4-oxo-7-(3-(dimethylamino)propoxy)-4H-quinolin-1-yl)-N-methyl-N-(4-(4-trifluoromethylphenyl)benzyl)acetamide bitartrate;
N-methyl-2-(2-(2-(2,3-difluoro-phenyl)ethyl)-7-nitro-4-oxo-4H-quinolin-1-yl)-N-(4-(4-trifluoro-methylphenyl)benzyl) acetamide;
2-(2-(2-(2,3-difluorophenyl)ethyl)-7-(2-dimethylaminoethoxy)-4-oxo-4H-quinolin-1-yl)-N-methyl-N-(4-(4-trifluoromethylphenyl)benzyl)acetamide bitartrate;
2-(7-(2-diethylaminoethoxy)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(2-(2-(2,3-difluorophenyl)ethyl)-4-oxo-7-(2-(piperidin-1-yl)ethoxy)-4H-quinolin-1-yl)-N-methyl-N-(4-(4-trifluoromethylphenyl)benzyl)acetamide bitartrate;
2-(2-(2-(2,3-difluorophenyl)ethyl)-4-oxo-7-(3-(piperidin-1-yl)propoxy)-4H-quinolin-1-yl)-N-methyl-N-(4-(4-trifluoromethylphenyl)benzyl)acetamide bitartrate;
tert. butyl 2-(2-(2-(2,3-difluorophenyl)ethyl))-1-(N-(4-(4-trifluoromethylphenyl)benzyl)-N-methyl-aminocarbonylmethyl)-4-oxo-4H-quinolin-7-yloxy)acetate;
2-(2-(2-(2,3-difluorophenyl)ethyl)-4-oxo-7-(pyridin-2-ylmethoxy)-4H-quinolin-1-yl)-N-methyl-N-(4-(4-trifluoromethylphenyl)benzyl)acetamide hydrochloride;
2-(2-(2-(2,3-difluorophenyl)ethyl)-7-(5-methylisoxazol-3-ylmethoxy)-4-oxo-4H-quinolin-1-yl)-N-methyl-N-(4-(4-trifluoromethylphenyl)benzyl)acetamide;
2-(2-(2-(2,3-difluorophenyl)ethyl)-7-(1-methylpyrrolidin-2-ylmethoxy)-4-oxo-4H-quinolin-1-yl)-N-methyl-N-(4-(4-trifluoromethylphenyl)benzyl)acetamide bitartrate;
2-(7-benzyloxy-2-(2-(2,3-difluorophenyl)ethyl)-4-oxo-4-quinolin-1-yl)-N-methyl-N-(4-(4-trifluoromethylphenyl)benzyl)acetamide;
2-(7-benzyloxy-2-(2-(2,3-difluorophenyl)ethyl)-4-oxo-4-quinolin-1-yl)-N-(1-ethyl-piperidin-4-yl)-N-(4-(4-trifluoromethylphenyl)benzyl)acetamide bitartrate;
2-(2-(2-(2,3-difluorophenyl)ethyl)-7-hydroxy-4-oxo-4H-quinolin-1-yl)-N-methyl-N-(4-(4-trifluoromethylphenyl)benzyl)acetamide;
2-(2-(2-(2,3-difluoro-phenyl)-ethyl)-7-hydroxy-4-oxo-4H-quinolin-1-yl)-N-(1-ethyl-piperidin-4-yl)-N-(4-(4-trifluoro-methyl-phenyl)benzyl)-acetamide bitartrate;
2-(2-(2-(2,3-difluorophenyl)ethyl))-1-(N-(4-(4-trifluoromethylphenyl)benzyl)-N-methyl-aminocarbonylmethyl)-4-oxo-4H-quinolin-7-yloxy)acetic acid
2-(7-(dimethylaminomethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(diethylaminomethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(diethylaminomethyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-(diethylaminomethyl)-2-(2-(2,3-difluorophenyl)ethyl)-4-oxo-4H-quinolin-1-yl)-N-isopropyl-N-(4-(4-trifluoromethylphenyl)benzyl)acetamide bitartrate;
2-(7-((pyrrolidin-1-yl)methyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-((piperidin-1-yl)methyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-(2-dimethylaminoethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(2-diethylaminoethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(2-(pyrrolidin-1-yl)ethyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(3-diethylaminopropyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(3-(pyrrolidin-1-yl)propyl)-2-(2-(2,3-difluorophenyl)ethyl)-4-oxo-4H-quinolin-1-yl)-N-methyl-N-(4-(4-trifluoromethylphenyl)benzyl)acetamide bitartrate;
2-(7-(3-(piperidin-1-yl)propyl)-2-(2-(2,3-difluorophenyl)ethyl)-4-oxo-4H-quinolin-1-yl)-N-ethyl-N-(4-(4-trifluoromethylphenyl)benzyl)acetamide bitartrate;
2-(7-(3-(piperidin-1-yl)propyl)-2-(2-(2,3-difluorophenyl)ethyl)-4-oxo-4H-quinolin-1-yl)-N-isopropyl-N-(4-(4-trifluoromethylphenyl)benzyl)acetamide bitartrate;
b 2-(7-(diethylaminomethyl)-2-(2,3-difluorobenzylthio)-4-oxo-4H-quinolin-1-yl)-N-methyl-N-(4-(4-trifluoromethylphenyl)benzyl)acetamide bitartrate; or
2-(7-(3-(4-morpholino)propyl)-2-(2,3-difluorobenzylthio)-4-oxo-4H-quinolin-1-yl)-N-methyl-N-(4-(4-trifluoromethylphenyl)benzyl)acetamide hydrochloride;
or the free base thereeof or another pharmaceutically salt thereof.

11. A pharmaceutical composition comprising a compound of formula (I) as claimed in claim 1 and a pharmaceutically acceptable carrier.

12-13. (canceled).

14. A method of treating a disease state associated with activity of the enzyme Lp-PLA₂ which method involves treating a patient in need thereof with a therapeutically effective amount of a compound of formula (I) as claimed in claim 1.

15. A process for preparing a compound of formula (I) as defined in claim 1 which process comprises reacting an acid compound of formula (II): 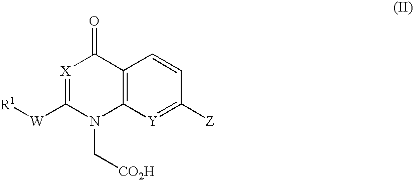
in which
R¹ is an aryl group, optionally substituted by 1, 2, 3 or 4 substituents which may be the same or different selected from the group consisting of C₍₁₋₆₎alkyl, C₍₁₋₆₎alkoxy, C₍₁₋₆₎alkylthio, hydroxy, halogen, CN, mono to perfluoro-C₍₁₋₄₎alkyl, mono to perfluoro-C₍₁₋₄₎alkoxyaryl, and arylC₍₁₋₄₎alkyl;
W is a C₍₂₋₄₎alkylene group, optionally substituted by 1, 2 or 3 substituents selected from the group consisting of methyl and ethyl, CH═CH, (CH₂)ₙS or (CH₂)ₙO where n is 1, 2 or 3;
X and Y are independently CH or N;
Z is NO₂, NR⁵R⁹, OR⁹, SR⁹, SOR⁹, SO₂R⁹ or R¹⁰;
R⁵ and R⁶ are independently hydrogen or C₍₁₋₁₂₎alkyl,
R⁷ and R⁸ may be the same or different and are hydrogen, or C₍₁₋₁₂₎alkyl, or R⁷ and R⁸ together with the nitrogen to which they are attached form a 5- to 7 membered ring optionally containing one or more further heteroatoms selected from the group consisting of oxygen, nitrogen and sulphur, and optionally substituted by one or two substituents selected from the group consisting of hydroxy, oxo, C₍₁₋₄₎alkyl, C₍₁₋₄₎alkylcarboxy, aryl, or aralkyl;
R⁹ is hydrogen or C₍₁₋₆₎alkyl optionally substituted by 1, 2 or 3 substituents which may be the same or different selected from the group consisting of hydroxy, halogen, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents selected from the group consisting of hydroxy, halogen, OR⁵, COR⁵, carboxy, COOR⁵, CONR⁷R⁸ and NR⁷R⁸; and
R¹⁰ is C₍₁₋₆₎alkyl optionally substituted by 1, 2 or 3 substituents which may be the same or different and is selected from the group consisting of hydroxy, halogen, OR⁵, COR⁵, COOR⁵, CONR⁷R⁸, NR⁷R⁸, NR⁵COR⁶, aryl, heteroaryl and a 5- to 7-membered heterocyclyl ring, which aryl or heteroaryl is optionally substituted by 1, 2, 3 or 4 substituents which may be the same or different and is selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁵, COOR⁵, CONR⁷R⁸, NR⁷R⁸, NR⁵COR⁶, SO₂NR⁷R⁸, NR⁵SO₂R⁶, mono to perfluoroC₍₁₋₄₎alkyl and mono to perfluoroC₍₁₋₄₎alkoxy, and which 5- to 7-membered heterocyclyl ring is optionally substituted by COR⁵, COOR⁵, CONR⁷R⁸, or C₍₁₋₆₎alkyl optionally substituted by 1, 2 or 3 substituents and is selected from the group consisting of hydroxy, halogen, OR⁵, COR⁵, carboxy, COOR⁵, CONR⁷R⁸ or NR⁷R⁸;
with an amine compound of formula (III):
R⁴—R³—CH₂NHR² (III)
in which R², R³ and R⁴ are
R² is hydrogen, C₍₁₋₆₎alkyl which may be unsubstituted or substituted by 1, 2 or 3 substituents selected from the group consisting of hydroxy, halogen, OR⁵, COR⁵, carboxy, COOR⁵, CONR⁷R⁸, NR⁷R⁸, NR⁵COR⁶, mono- or di-(hydroxyC₍₁₋₆₎alkyl)amino and N-hydroxyC₍₁₋₆₎alkyl-N—C₍₁₋₆₎alkylamino; or
R² is Het-C₍₀₋₄₎alkyl in which Het is a 5- to 7-membered heterocyclyl ring comprising N and optionally O or S, and in which N may be substituted by COR⁵, COOR⁵, CONR⁷R⁸, or C₍₁₋₆₎alkyl optionally substituted by 1, 2 or 3 substituents selected from the group consisting of hydroxy, halogen, OR⁵, COR⁵, carboxy, COOR⁵, CONR⁷R⁸ and NR⁷R⁸;
R³ is an aryl or a heteroaryl ring optionally substituted by 1, 2, 3 or 4 substituents which may be the same or different selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁵, carboxy, COOR⁵, NR⁵COR⁶, CONR⁷R⁸, SO₂NR⁷R⁸, NR⁵SO₂R⁶, NR⁷R⁸, mono to perfluoro-C₍₁₋₄₎alkyl and mono to perfluoro-C₍₁₋₄₎alkoxy;
R⁴ is an aryl or a heteroaryl ring which is optionally substituted by 1, 2, 3 or 4 substituents which may be the same or different selected from the group consisting of C₍₁₋₆₎alkyl, C₍₁₋₆₎alkoxy, C₍₁₋₆₎alkylthio, C₍₁₋₆₎alkylsulfonyl, arylC₍₁₋₆₎alkoxy, hydroxy, halogen, CN, COR⁵, carboxy, COOR⁵, CONR⁷R⁸, NR⁵COR⁶, SO₂NR⁷R⁸, NR⁵SO₂R⁶, NR⁷R⁸, mono to perfluoro-C₍₁₋₄₎alkyl and mono to perfluoro-C₍₁₋₄₎alkoxy, or C₍₅₋₁₀₎alkyl;
under amide forming conditions.
